(19) United States
(12) Patent Application Publication
(10) Pub. No.: US 2013/0136805 A1
(43) Pub. Date: May 30, 2013

(54) FOAMABLE COMPOSITIONS OF STABILIZED CHLORITE

(71) Applicants: Servet Buyuktimkin, San Diego, CA (US); Nadir Buyuktimkin, San Diego, CA (US); Edward T. Kisak, San Diego, CA (US); John M. Newsam, La Jolla, CA (US); Dominic King-Smith, San Diego, CA (US); Jagat Singh, Toronto (CA); Rainer Martin, Waghaeusel (DE)

(72) Inventors: Servet Buyuktimkin, San Diego, CA (US); Nadir Buyuktimkin, San Diego, CA (US); Edward T. Kisak, San Diego, CA (US); John M. Newsam, La Jolla, CA (US); Dominic King-Smith, San Diego, CA (US); Jagat Singh, Toronto (CA); Rainer Martin, Waghaeusel (DE)

(21) Appl. No.: 13/816,056

(22) PCT Filed: Aug. 12, 2011
(86) PCT No.: PCT/CA2011/000914

§ 371 Date: Feb. 8, 2013

Related U.S. Application Data
(60) Provisional application No. 61/373,467, filed on Aug. 13, 2010.

(51) Int. Cl. A61K 9/12

(52) U.S. Cl. CPC A61K 9/122
USPC 424/661

(57) ABSTRACT

The present application relates to foamable compositions and foams comprising stabilized chlorite, including stabilized chlorite solutions such as Oxovasin™, and to medical uses thereof, in particular for wound healing. The foamable compositions comprise at least one foaming agent, stabilized chlorite, water and optionally, a buffering agent.

### RELATED APPLICATIONS

The present application claims the benefit of priority of co-pending U.S. provisional patent application No. 61/373,467 filed on Aug. 13, 2010, the contents of which are incorporated herein by reference in their entirety.

### FIELD OF THE APPLICATION

The present application relates to foamable compositions comprising stabilized chlorite, methods for their preparation and their topical use, for example, for wound healing.

### BACKGROUND OF THE APPLICATION

### Topical Foams

A foam is a multiphase mixture comprising bubbles of a gas phase that are separated by a liquid or solid layer (a film) (Pilpel N., Foams in pharmacy, Endeavour 9: 87-91 (1985); Durian, D. J. and Weitz, D. A., “Foams” in Kirk-Othmer Encyclo. Chem. Tech., 4th ed., 11: 783-805 (1994)). Liquid-based foams are dynamic systems that eventually collapse or break to release the gas phase. A foam's collapsibility or breakability depends on numerous physical properties of its components, such as the liquid phase's viscosity and surface tension, the gas phase's pressure and bubble size, and the film's elasticity.

To persist for more than a short period, a foam preferably includes at least one foaming agent such as a protein or surfactant. Surfactants stabilize the foam, e.g., by inhibiting bubble coalescence (Zhao, Y.; Brown, M. B.; Jones, S. J., Pharmaceutical foams: are they the answer to the dilemma of topical nanoparticles?, Nanomedicine, in press (2010)).

As discussed in WO2009/090558, the types of topical foam vehicles include aqueous foams, such as commonly available shaving foams; hydroalcoholic foams; emulsion-based foams, comprising oil and water components; and oleaginous foams, which comprise high levels of oil. When a foam is applied to the skin or to a wound bed, lower alcohol compounds in the formulation may increase penetration, but may also dry the skin and may cause stinging if applied to wounds or sores.

Some foams are long-lasting (e.g., shaving creams or shaving foams formed from gels). Other foams are quick-breaking and collapse soon after application, which can allow more rapid absorption of an active agent in the foam. However, if the foam breaks too quickly, it will be difficult to apply. Quick-breaking foams may be destabilized by body heat (thermolabile) or by force (labile to mechanical stress), which allows easy spreading over the site of application.

Mere combination of basic ingredients does not automatically produce foams suitable for pharmaceutical or cosmetic use. Small changes in the foam base, such as the addition of active ingredients or co-solvents, may destabilize a foam. Similarly, selection within a group of ingredients may provide a foam or class of foams that provides unpredictably superior properties.

Although a foam's properties can be difficult to predict, properties such as collapsibility or stiffness are factors to consider for the foam's intended use. For example, a pharmaceutical foam for internal application may desirably persists for hours or days to release an active agent slowly. A pharmaceutical foam for topical application to skin usually desirably breaks down more quickly, but not so quickly that the liquid or solid phase will drip off the skin before absorption of sufficient active agent.

Qualities such as foam stability, ease of spreading and appropriate breakability upon application to the skin or wound are desirable features. These characteristics can be measured by conducting foam formation and foam collapsibility experiments. Foam formation (foam height vs time), for example, may be predictive of the generation of a sprayable/spreadable foam. The rate of collapse is a property relevant to the appropriate administration of the foam.

Many foams are generated by dispensing a foam base in combination with a dissolved, gaseous propellant that expands upon release from a container to produce the foam's bubbles. However, manufacturing a propellant-based composition can be costly and difficult, and the associated canisters can harm the environment. It is therefore desirable to develop a composition that is foamable in the absence of a propellant.

Pharmaceutical foams have been used in wound dressings, contraception, and topical drug delivery. They can be easy to apply uniformly to skin, less messy than cream or liquid dosage forms, and less irritating to sensitive or abraded skin. Zhao, Y. et al., Id. The superior properties of foams may enhance patient compliance. The dispensing means of a foam composition can help to prevent contamination of the container during application. For at least these reasons, foams are attractive dosage forms for topically absorbable active agents applied for treating wounds.

### Wound Treatments

Chronic, hard-to-heal wounds are a serious problem with an increasing incidence. Chronic wounds can be caused by such conditions as pressure sores and poor circulation in the lower extremities. Co-morbid conditions, such as diabetes and atherosclerosis, reduce blood flow to the extremities and can also increase the likelihood of developing chronic wounds.

The use of an aqueous solution containing a stabilized chlorite solution for treating wounds and infections is described in Hinz et al., The Lancet (1986), U.S. Pat. Nos. 4,507,285 and 4,725,437, and EP 0 200 157. These documents describe the use of a stabilized chlorite solution in stimulating the wound healing response in humans, as well as in treating infections caused by parasites, fungi, bacteria, viruses and/or mycoplasma. Kuhne et al., European Patent No. 200,156, describes the use of a stabilized chlorite solution in conjunction with radiation therapy to aid in repairing damaged irradiated tissue and reducing side effects.

It has been proposed that a macrophage is stimulated by the stabilized chlorite solution. In the presence of heme compounds (e.g., hemoglobin, myoglobin, peroxidases, cytochromes, etc.), which are present in the serum which also are part of the cell membrane of phagocytic cells like macrophages, the stabilized chlorite solution becomes a secondary oxidant with oxidative properties different from chlorite and hydrogen peroxide.

It has further been proposed that the known wound-healing mechanism via macrophage activation by the stabilized chlorite solution also stimulates and enhances the phagocytic activity of the macrophage. Thus, the activated macrophage is primed to ingest, digest and dispose of foreign antigens. The use of a stabilized chlorite solution to render a macrophage phagocytic is described in EP 0 200 157.

The antimicrobial properties of stabilized chlorite solutions is well documented in the scientific literature (see, for example, Teepe, R. C. G., Koebrugge, E. J., Löwik, C. W. G. M., Petit, P. L. C., Bosboom, R. W., Twiss, I. E., Boxma, H., Vermeer, B. J., Ponec, M. J. of Trauma Vol. 35, No. 1, 8-19 (1993)).

### SUMMARY OF THE APPLICATION

The present application relates to foamable compositions and methods of using foamable compositions, for example, to treat wounds. In particular the present application relates to a topical composition, such as a stabilized chlorite foam, for use in wound healing. The challenge has been to develop a composition that will deliver the active agent topically in sufficient concentration to treat a wound, possibly on a long-term basis, while still providing a foam with an appropriate collapsibility or breakability. A foamable composition of a stabilized chlorite solution will provide improved administration of its active ingredient in addition to the other advantages of a foam, including, for example, providing a non-runny topical dosage form that can be conveniently distributed across a wound. However, none of the commercially available stabilized chlorite solutions tested, form an appreciable foam on their own.

The stabilized chlorite solutions of the present application comprise aqueous compositions containing a therapeutically effective amount of chlorite, wherein the pH and activity of the solution is stable. Non-limiting examples of commercially available stabilized chlorite solutions include WF10, Oxovasin™, and OXO-K993. Other non-limiting stable chlorite-based solutions are described in, for example, U.S. Pat. Nos. 6,350,438, 6,251,372, 6,235,269, 6,132,702, 6,077,502 and 4,574,084.

In an embodiment of the application, the foamable composition comprises: (i) stabilized chlorite; (ii) at least one foaming agent; (iii) water; and, optionally, (iv) a buffering agent.

In another embodiment of the application, the foamable composition further comprises at least one foam stabilizer.

In another embodiment, the foamable composition comprises: (i) about 0.1% to about 10% (w/w) of stabilized chlorite; (ii) about 0.5% to about 5% (w/w) of at least one foaming agent; (iii) about 0.0% to about 1.0% (w/w) of a buffering agent; and (iv) water (q.s.). In a further embodiment, the foamable composition comprises: (i) about 0.5% to about 3% (w/w) of stabilized chlorite; (ii) about 1% to about 3% (w/w) of at least one foaming agent; (iii) about 85% to about 99% (w/w) water; and (iv) about 0.0% to about 0.2% (w/w) of a buffering agent. In a further embodiment, the stabilized chlorite is present in the composition in an amount of about 1.5% to about 2.5% (w/w); the at least one foaming agent is present in the composition in an amount of about 1.5% to about 2.5% (w/w); water is present in an amount of about 90% to about 99% (w/w); and the buffering agent is present in an amount of about 0.05% to about 0.15% (w/w). In a further embodiment the at least one foaming agent includes, in addition to the foaming agent, at least one foam stabilizer.

In another embodiment, the stabilized chlorite is a composition comprising chlorite, such as OXO-K993, or the stabilized chlorite and water are combined to form a diluted solution of chlorite, such as a dilution solution of OXO-K993, (or a “stabilized chlorite solution”), for example, a 1-10%, a 10-20%, a 20-30%, a 30-50% or a 50-90% (w/v) diluted solution of OXO-K993. In an embodiment, the stabilized chlorite and water are combined to form a 2% (w/v) solution of OXO-K993. In a further embodiment, the stabilized chlorite solution is a solution (Oxovasin™) comprising about 2% (w/v) OXO-K993, about 2% (w/v) glycerol and about 96% (w/v) water. In another embodiment, the stabilized chlorite solution is present in the composition in an amount of 97.25, 97.35, 97.45, 97.55, 97.65, 97.75, 97.85, 97.95, 98.05, 98.15, 98.25, 98.35, 98.45, 98.55, 98.65, 98.75, 98.85 or 98.95% (w/w), or fractions in between.

In a further embodiment of the present application, the foamable composition comprises about 0.01% (w/w) to about 1.0% (w/w), about 0.02% (w/w) to about 0.5% (w/w), about 0.05% (w/w) to about 0.20% (w/w) or about 0.08% (w/w) to about 0.1% (w/w) of chlorite ion (ClO₂⁻).

In a further embodiment, the foaming agent and/or foam stabilizer is selected from poloxamer copolymers, polyvinylalcohol (PVA), polyvinylpyrrolidone (PVP), cellulose ethers, polysorbates (Tween™), coco-derived amphoteric surfactants, and combinations thereof. In an embodiment, the at least one foaming agent is present in the composition in an amount of 1.15, 1.25, 1.35, 1.45, 1.55, 1.65, 1.75, 1.85, 1.95, 2.05, 2.15, 2.25, 2.35, 2.45, 2.55, 2.65 or 2.75% (w/w), or fractions in between. In a further embodiment, the at least one foaming agent includes, in addition to the foaming agent, at least one foam stabilizer.

In an embodiment, the foaming agent and, if present, the foam stabilizer, comprises, consists or consists essentially of a poloxamer, a cellulose ether and, optionally, PVA. In another embodiment, the foaming agent and, if present, the foam stabilizer comprises, consists or consists essentially of a poloxamer, a cellulose ether and PVA. In an embodiment, the cellulose ether is hydroxypropyl cellulose.

In an embodiment, the buffering agent is an inorganic base. In a further embodiment, the buffering agent is present in the composition in an amount of 0.005, 0.015, 0.025, 0.035, 0.045, 0.055, 0.065, 0.075, 0.085 or 0.095% (w/w), or fractions in between. In one embodiment, the inorganic base is sodium carbonate.

In an embodiment, the foamable composition is propellant-free.

In another embodiment, the foamable composition has a pH of about 10 or above.

In an embodiment of the application, the foamable composition when foamed does not collapse to a liquid phase for at least 30 seconds at 37° C. or at skin temperature. In a further embodiment, the composition when foamed does not collapse to a liquid phase for at least 60 seconds at 37° C. or at skin temperature. In yet another embodiment, the composition when foamed does not collapse to a liquid phase for at least two minutes at 37° C. or at skin temperature. In a further embodiment, the composition when foamed does not collapse to a liquid phase for at least five minutes at 37° C. or at skin temperature.

The application also includes a dispenser comprising a reservoir operably linked (e.g., in fluid communication) with a release assembly, wherein the reservoir contains a foamable composition of the application, and wherein the release assembly allows the foamable composition to be released as a foam. In an embodiment, the release assembly is a dispensing head.

The application also includes a pressurized container, the container holding a foamable composition of the present application and optionally a propellant. In an embodiment, a release assembly comprising a nozzle or sprayer is operably linked to the container (e.g., a release assembly for dispensing a foam from an aerosol spray can wherein the release assembly is in fluid communication with the spray can), wherein the release assembly allows the foamable composition and optionally a propellant to be released as a foam.

The application also includes a use of the compositions of the present application as a medicament or antimicrobial agent.

The application further includes a method for treating wounds comprising applying an effective amount of a foamable composition of the application to a subject in need thereof. In one embodiment, the foamable composition is applied to the skin of the subject. In another embodiment, the foamable composition is applied to the wound bed of a subject.

The application further includes a use of a foamable composition of the application for wound healing, including pressure, post-operative or post-traumatic wound healing, or chronic wound healing as in the healing of diabetic ulcers, venous ulcers, arterial ulcers or decubitus ulcers.

The present application also includes a foam comprising a foamable composition of the application.

Other features and advantages of the present application will become apparent from the following detailed description. It should be understood, however, that the detailed description and the specific examples, while indicating embodiments of the application, are given by way of illustration only, since various changes and modifications within the spirit and scope of the application will become apparent to those skilled in the art from this detailed description.

### BRIEF DESCRIPTION OF THE DRAWINGS

The embodiments of the application will now be described in greater detail with reference to the attached drawings in which:

FIG. 1 shows bar graphs illustrating the foam quality of compositions Oxo100406-01 through Oxo100406-09 (a) after spraying and (b) after spreading (refer to Table 1 for ingredient details). In (a) 5=no leak; 4=light leak; and 3=leak. In (b) 5=foam; 4=light foam; 3=lighter foam; 2=very light foam; and 1=no foam.

FIG. 2 shows bar graphs illustrating the foam quality of compositions Oxo100419-01 through Oxo100419-07 (a) after spraying and (b) after spreading (refer to Table 2 for ingredient details). In (a) 5=no leak; 4=light leak; and 3=leak. In (b) 5=foam; 4=light foam; 3=lighter foam; 2=very light foam; and 1=no foam.

FIG. 3 shows bar graphs illustrating the foam quality of compositions Oxo100420-01 through Oxo100420-07 (a) after spraying and (b) after spreading (refer to Table 3 for ingredient details). In (a) 5=no leak; 4=light leak; and 3=leak. In (b) 5=foam; 4=light foam; 3=lighter foam; 2=very light foam; and 1=no foam.

FIG. 4 shows bar graphs illustrating the foam quality of compositions Oxo100427-01 through Oxo100427-07 (a) after spraying and (b) after spreading (refer to Table 4 for ingredient details). In (a) 5=no leak; 4=light leak; and 3=leak. In (b) 5=foam; 4=light foam; 3=lighter foam; 2=very light foam; and 1=no foam.

FIG. 5 shows bar graphs illustrating the foam quality of compositions Oxo100430-01 through Oxo100430-07 (a) after spraying and (b) after spreading (refer to Table 5 for ingredient details). In (a) 5=no leak; 4=light leak; and 3=leak. In (b) 5=foam; 4=light foam; 3=lighter foam; 2=very light foam; and 1=no foam.

FIG. 6 shows bar graphs illustrating the stability of foam quality of compositions F1 and F2. FIG. 6 (a) illustrates the foam height at 0 and 2 minutes following shaking of composition F1 taken from samples stored for 0, 1, 2, 3, 4, 8, 12, 16, 20, and 24 weeks. FIG. 6 (b) illustrates the foam height at 0 and 2 minutes following shaking of composition F2 taken from samples stored for 0, 1, 2, 3, 4, 8, 12, 16, 20, and 24 weeks.

FIG. 7 shows bar graphs illustrating the stability of foam quality of compositions F3 and F4. FIG. 7 (a) illustrates the foam height at 0 and 2 minutes following shaking of composition F3 taken from samples stored for 0, 1, 2, 3, 4, 8, 12, 16, 20, and 24 weeks. FIG. 7 (b) illustrates the foam height at 0 and 2 minutes following shaking of composition F4 taken from samples stored for 0, 1, 2, 3, 4, 8, 12, 16, 20, and 24 weeks.

FIG. 8 shows bar graphs illustrating the stability of foam quality of compositions F5 and F6. FIG. 8 (a) illustrates the foam height at 0 and 2 minutes following shaking of composition F5 taken from samples stored for 0, 1, 2, 3, 4, 8, 12, 16, 20, and 24 weeks. FIG. 8 (b) illustrates the foam height at 0 and 2 minutes following shaking of composition F6 taken from samples stored for 0, 1, 2, 3, 4, 8, 12, 16, 20, and 24 weeks.

FIG. 9 shows bar graphs illustrating the stability of foams: (a) according to their pH profiles following 0, 1, 2, 3, 4, 8, 12, 16, 20, and 24 weeks of storage; and (b) according to HPLC analysis of their chlorite concentration following 16, 20, and 24 weeks of storage.

FIG. 10 shows bar graphs illustrating the pH change of composition Ox100520-02 over 20 weeks: (a) in standard containers; and (b) in the high density polyethylene (“HDPE”) container used for commercially-available Oxovasin™.

FIG. 11 shows a bar graph illustrating the stability of composition Ox100520-03 according to its pH profile following 1, 2, 3, 4, 8, 12, 16 and 20 weeks of storage.

FIG. 12 shows a bar graph illustrating the stability of composition Ox100520-04 according to its pH profile following 1, 2, 3, 4, 8, 14, 16 and 20 weeks of storage.

FIG. 13 shows bar graphs illustrating the stability of foam composition Ox100520-02. FIG. 13 (a) illustrates the foam height at 2 minutes following shaking of the initial samples and compositions stored for 1, 2, 3, 4, 8, 14, 16, 20 and 24 weeks. FIG. 13 (b) illustrates the foam height at 2 minutes following shaking of the initial samples and compositions stored in a commercial Oxavasin container closure for 1, 2, 3, 4, 8, 14, 16, 20 and 24 weeks. In (b), the sample was transferred to a 4 dram glass vial before the shake height assay was conducted.

FIG. 14 shows a bar graph illustrating the stability of foam composition Ox100520-03. The foam height at 2 minutes following shaking of composition Ox100520-03 taken from initial samples and those stored for 1, 2, 3, 4, 8, 14, 16, 20, and 24 weeks was measured.

FIG. 15 shows a bar graph illustrating the stability of foam composition Ox100520-04. The foam height at 2 minutes following shaking of composition Ox100520-04 taken from initial samples and those stored for 1, 2, 3, 4, 8, 14, 16, 20, and 24 weeks was measured.

FIG. 16 shows a bar graph illustrating the stability of foams in the Ox100520 series according to HPLC analysis of their chlorite concentrations following 0, 1, 2, 3, 8, 12, 16, 20, and 24 weeks of storage.

FIG. 17 shows a bar graph illustrating the stability of composition Ox100712-01 according to its pH profile following 1, 2, 3, 4, 10, 12 and 16 weeks of storage.

FIG. 18 shows a bar graph illustrating the stability of composition Ox100712-02 according to its pH profile following 1, 2, 3, 4, 10, 12 and 16 weeks of storage.

FIG. 19 shows a bar graph illustrating the stability of composition Ox100712-03 according to its pH profile following 1, 2, 3, 4, 9, 12 and 16 weeks of storage.

FIG. 20 shows a bar graph illustrating the stability of composition Ox100712-04 according to its pH profile following 1, 2, 3, 4, 9, 12 and 16 weeks of storage.

FIG. 21 shows a bar graph illustrating the stability of composition Ox100712-05 according to its pH profile following 1, 2, 3, 4, 8 and 12 weeks of storage.

FIG. 22 shows a bar graph illustrating the stability of foam composition Ox100712-01. The foam height at 2 minutes following shaking of composition Ox100712-01 taken from initial samples and those stored for 1, 2, 3, 4, 12, and 16 weeks was measured.

FIG. 23 shows a bar graph illustrating the stability of foam composition Ox100712-02. The foam height at 2 minutes following shaking of composition Ox100712-02 taken from initial samples and those stored for 1, 2, 3, 4, 8, 10, 12, and 16 weeks was measured.

FIG. 24 shows a bar graph illustrating the stability of foam composition Ox100712-03. The foam height at 2 minutes following shaking of composition Ox100712-03 taken from initial samples and those stored for 1, 2, 3, 4, 9, 12, and 16 weeks was measured.

FIG. 25 shows a bar graph illustrating the stability of foam composition Ox100712-04. The foam height at 2 minutes following shaking of composition Ox100712-04 taken from initial samples and those stored for 1, 2, 3, 4, 9, 12, and 16 weeks was measured.

FIG. 26 shows a bar graph illustrating the stability of foam composition Ox100712-05. The foam height at 2 minutes following shaking of composition Ox100712-05 taken from initial samples and those stored for 1, 2, 3, 8, and 12, weeks was measured.

FIG. 27 shows a bar graph illustrating the stability of foams in the Ox100712 series according to HPLC analysis of their chlorite concentration following 1, 2, 3, 4, 8, 10, 12 and 16 weeks of storage.

FIG. 28 shows a bar graph illustrating the stability of composition Ox100802-01 according to its pH profile following 1, 2, 3, 8 and 12 weeks of storage.

FIG. 29 shows a bar graph illustrating the stability of composition Ox100802-02 according to its pH profile following 1, 2, 3, 8 and 12 weeks of storage.

FIG. 30 shows a bar graph illustrating the stability of composition Ox100802-03 according to its pH profile following 1, 2, 3, 8 and 12 weeks of storage.

FIG. 31 shows a bar graph illustrating the stability of foam composition Ox100802-01. The foam height at 2 minutes following shaking of composition Ox100802-01 taken from initial samples and those stored for 1, 2, 3, 8, and 12, weeks was measured.

FIG. 32 shows a bar graph illustrating the stability of foam composition Ox100802-02. The foam height at 2 minutes following shaking of composition Ox100802-02 taken from initial samples and those stored for 1, 2, 3, 8, and 12, weeks was measured.

FIG. 33 shows a bar graph illustrating the stability of foam composition Ox100802-03. The foam height at 2 minutes following shaking of composition Ox100802-03 taken from initial samples and those stored for 1, 2, 3, 8, and 12, weeks was measured.

FIG. 34 shows a bar graph illustrating the stability of foams in the Ox100802 series according to HPLC analysis of their chlorite concentration following 0, 1, 2, 3, 8, 12 and 16 weeks of storage.

### DETAILED DESCRIPTION OF THE APPLICATION

### I. Definitions

Unless otherwise indicated, the definitions and embodiments described in this and other sections are intended to be applicable to all embodiments and aspects of the application herein described for which they are suitable as would be understood by a person skilled in the art.

The terms “a,” “an,” or “the” as used herein not only include aspects with one member, but also includes aspects with more than one member. For example, an embodiment including “a surfactant” should be understood to present certain aspects with one surfactant or two or more additional surfactants.

Terms of degree such as “about”, and “approximately” and “substantially” as used herein mean a reasonable amount of deviation of the modified term such that the end result is not significantly changed. These terms of degree should be construed as including a deviation of at least ±5% of the modified term if this deviation would not negate the meaning of the word it modifies.

The term “q.s.” as used herein means “quantum sufficiat”, or the quantity sufficient to provide the desired volume. Generally, this means the quantity of water to make the total ingredients in a composition equal to 100%.

In compositions comprising an “additional” or “second” component, the second component as used herein is chemically different from the other components or first component. A “third” component is different from the other, first, and second components, and further enumerated or “additional” components are similarly different.

The term “agent” as used herein indicates a compound or mixture of compounds that, when added to a composition, tend to produce a particular effect on the composition's properties.

The term “stabilized chlorite” as used herein refers to a composition or substance, comprising chlorite ions (ClO₂⁻) and in which the concentration of chlorite ions, the pH and/or the activity remains stable for an acceptable period of time prior to use. In a stabilized chlorite, the chlorite ions do not substantially degrade and the activity of the chlorite ions is substantially maintained prior to use. The concentration of chlorite ions may be monitored, for example, by monitoring the pH or by high performance liquid chromatography (HPLC).

The term “an acceptable period of time” as used herein means at least about 1 day, at least about 1 week, at least about 30 days, at least about six months, at least about one year, at least about two years, or at least about the time between preparation and use.

The term “foamable composition” as used herein refers to an aqueous solution comprising stabilized chlorite, foaming agent(s) and optional other ingredients (for example, buffering agent(s)) prior to conversion of the solution to a foam.

The term OXO-K993 as used herein refers to an aqueous solution containing sodium chlorite (about 4.25% w/w), sodium chloride (about 1.9% w/w), sodium chlorate (1.5% w/w) and sodium sulfate (0.7% w/w). OXO-K993 is also referred to in the art as tetrachlorodecaoxide (TCDO).

The term “alkali metal base” as used herein refers to a basic substance that comprises an inorganic or organic anion and an alkali metal cation and includes, for example, sodium hydroxide, potassium hydroxide, lithium hydroxide, calcium hydroxide, pentasodium triphosphate, potassium pyrophosphate, sodium pyrophosphate, sodium citrate, potassium citrate, sodium carbonate, potassium carbonate, lithium carbonate, sodium bicarbonate, lithium bicarbonate, calcium bicarbonate and potassium bicarbonate and mixtures thereof. The alkali metal base is also referred to in the art as an “alkali metal builder”.

The term “buffering agent” as used herein refers to a compound or mixture of compounds that adjusts the pH of the composition.

The term “cellulose ethers” refer to cellulose derivatives wherein the hydroxyl groups have been partially or completely converted into ether functional groups. Examples of cellulose ethers include cellulose, hydroxypropyl cellulose (“HPC”), hydroxypropyl methyl cellulose (“HPMC”), hydroxyethyl cellulose (“HEC”), methyl cellulose, carboxymethyl cellulose, carboxymethyl cellulose sodium salt and the like.

The term “coco-derived amphoteric surfactants” refers to a class of surfactants comprising a long hydrocarbon chain derived from coconut oils and includes, for example, sodium cocoamphoacetate, sodium cocoamphopropionate, disodium cocoamphodiacetate, disodium cocoamphodipropionate and the like.

“Composition” and “pharmaceutical composition” as used herein are equivalent terms referring to a composition of matter for pharmaceutical use.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “effective amount” as used herein means an amount sufficient to achieve the desired result and accordingly will depend on the ingredient and its desired result. Nonetheless, once the desired effect is known, determining the effective amount is within the skill of a person skilled in the art.

In general, the “error bars” on the graphs represent the standard error of the mean value, whereas the top of the solid, shaded bar represents a single data value, which is the mean value of the distribution of data values.

The term “foam” as used herein refers to a substance that is formed by trapping many gaseous bubbles in a liquid.

The term “foaming agent” as used herein refers to a compound that assists in the formation of a foam. Foaming agents can also act as foam stabilizers.

The term “foam stabilizers” refers to compounds that tend to enhance the integrity and persistence of the foam. Foam stabilizers can also act as foaming agents.

The term “HY117” as used herein refers to an HPC having an approximate mean molecular weight of 95,000 g/mol.

The term “inorganic base” as used herein refers to a basic substance that comprises an inorganic anion and cation. The inorganic base is suitably one that is soluble in water or aqueous solutions and is compatible with the other ingredients in the compositions of the application.

The term “pharmaceutically acceptable” means compatible with the treatment of animals, in particular, humans.

The term “poloxamer” as used herein refer to nonionic triblock copolymers composed of a central hydrophobic chain of polyoxypropylene (poly(propylene oxide)) flanked by two hydrophilic chains of polyoxyethylene (poly(ethylene oxide)).

“Poloxamer 124” as used herein refers to a poloxamer copolymer of the general formula HO(C₂H₄O)ₐ(C₃H₆O)_{b}(C₂H₄O)ₐH where a is approximately 12 and b is approximately 20.

“Poloxamer 188” as used herein refers to a poloxamer copolymer of the general formula HO(C₂H₄O)ₐ(C₃H₆O)_{b}(C₂H₄O)ₐH where a is approximately 80 and b is approximately 27. Poloxamer 188 is also known as Pluronic™ F68.

“Poloxamer 237” as used herein refers to a poloxamer copolymer of the general formula HO(C₂H₄O)ₐ(C₃H₆O)_{b}(C₂H₄O)ₐH where a is approximately 64 and b is approximately 37.

“Poloxamer 338” as used herein refers to a poloxamer copolymer of the general formula HO(C₂H₄O)ₐ(C₃H₆O)_{b}(C₂H₄O)ₐH where a is approximately 141 and b is approximately 44.

“Poloxamer 407” as used herein refers to a poloxamer copolymer of the general formula HO(C₂H₄O)ₐ(C₃H₆O)_{b}(C₂H₄O)ₐH where a is approximately 101 and b is approximately 56.

The term “polysorbate” as used herein refers to a class of polymer surfactants derived from pegylated sorbitan, esterified with fatty acids. A common class of polysorbates are the “Tween™” surfactants.

The term “polyvinyl alcohol” as used herein refers to a synthetic polymer comprising monomeric units of the formula:

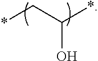

It is to be understood that often polyvinyl alcohol is incompletely hydrolyzed and its structure can be represented as

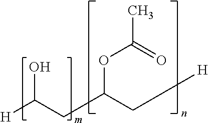

In an embodiment, the values of m and n fall in the range defined by 0.85≦m/(n+m)≦0.89 or 0≦n/m≦0.35.

The term “polyvinylpyrrolidone” as used herein refers to a polymer made from the monomer N-vinylpyrrolidone, comprising monomeric units of the formula:

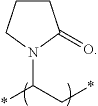

The term “PVP-30” as used herein refers to a polyvinylpyrrolidone polymer having an approximate mean molecular weight of 330,000 g/mol.

The term “subject” as used herein includes all members of the animal kingdom, including mammals, and suitably refers to humans.

“Topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

“Transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

“Transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The term “epicutaneous application” as used herein means administration directly on, or through, the skin.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “wound” as used herein refers to a type of injury or condition in which the dermis of the skin is damaged, or absent, including for example, infected wounds, pressure wounds, chronic wounds, delayed or problematic post-traumatic or post-op wound healing, decubitus ulcers, chronic leg ulcers in venous insufficiency, ulcers & wounds due to arterial blood flow, diabetic microangiopathy, diabetic ulcers, gangrene, psoriasis, atopic or neuro dermatitis and burns.

The term “treating wounds” or “wound healing” as used herein means to facilitate the contraction, closure and/or faster healing of wounds using the compositions of the present application, in particular compared to wounds treated in an identical fashion except in the absence of the compositions of the present application.

The term “water” as used herein as an ingredient in the compositions of the application refers to pharmaceutically acceptable water.

The term “w/v” means a percentage expressed in terms of weight of the ingredient or agent over the total volume of the composition multiplied by 100.

The term “w/w” means a percentage expressed in terms of weight of the ingredient or agent over the total weight of the composition multiplied by 100.

### II. Foamable Compositions

The foamable compositions of the present application are intended for topical use to locally promote wound healing. Without wishing to be limited by theory, the principal effect of these compositions may be due to their ability to activate phagocytosis in granulocytes and macrophages. It is known that the intracellular killing of bacteria during phagocytosis by granulocytes is dependent on O₂. This is especially important since wound healing is retarded by anaerobes and facultative anaerobes (such as occur in chronic leg ulcers for example). Macrophages have a dominant role in controlling inflammation and in regenerating damaged tissue, secreting mediator that favor angiogenesis and fibroblast proliferation.

It has surprisingly been found that a stable chlorite foam-containing composition can be made by combining aqueous stabilized chlorite with suitable foam-forming agents. Accordingly, in one embodiment of the application, there is included a foamable composition comprising: (i) stabilized chlorite; (ii) at least one foaming agent; (iii) water; and, optionally, (iv) a buffering agent.

In another embodiment of the application, the foamable composition further comprises at least one foam stabilizer.

In another embodiment, the foamable composition comprises: (i) about 0.1% to about 10% (w/w) of stabilized chlorite; (ii) about 0.5% to about 5% (w/w) of at least one foaming agent; (iii) about 0.0% to about 1.0% (w/w) of a buffering agent; and (iv) water (q.s.). In a further embodiment, the foamable composition comprises: (i) about 0.5% to about 3% (w/w) of stabilized chlorite; (ii) about 1% to about 3% (w/w) of at least one foaming agent; (iii) about 85% to about 98.5% (w/w) water; and (iv) about 0.0% to about 0.2% (w/w) of a buffering agent. In a further embodiment, the stabilized chlorite is present in the composition in an amount of about 1.5% to about 2.5% (w/w); the at least one foaming agent is present in the composition in an amount of about 1.5% to about 2.5% (w/w); water is present in an amount of about 90% to about 96.95% (w/w); and the buffering agent is present in an amount of about 0.05% to about 0.15% (w/w). In an embodiment, the at least one foaming agent, includes, in addition to the foaming agent, at least one foam stabilizer.

### A. Stabilized Chlorite

The foamable compositions of the present application comprise stabilized chlorite. The stabilized chlorite is a composition comprising a therapeutically effective amount of chlorite.

Non-limiting examples of stabilized chlorite-based compositions include those described in U.S. Pat. Nos. 6,350,438, 6,251,372, 6,235,269, 6,132,702, 6,077,502 and 4,574,084, the contents of each of which is incorporated by reference in their entirety.

In another embodiment, the stabilized chlorite is a composition comprising chlorite, such as OXO-K993 or the stabilized chlorite and water are combined to form a diluted solution of chlorite, such as a dilution solution of OXO-K993, (or a “stabilized chlorite solution”), for example, a 1-10%, a 10-20%, a 20-30%, a 30-50% or a 50-90% (w/v) diluted solution of OXO-K993. In an embodiment, the stabilized chlorite and water are combined to form a 2% (w/v) solution of OXO-K993. In a further embodiment, the stabilized chlorite solution is a solution (Oxovasin™) comprising about 2% (w/v) OXO-K993, about 2% (w/v) glycerol and about 96% (w/v) water. In another embodiment, the stabilized chlorite solution (i.e. stabilized chlorite plus water) is present in the composition in an amount of 97.25, 97.35, 97.45, 97.55, 97.65, 97.75, 97.85, 97.95, 98.05, 98.15, 98.25, 98.35, 98.45, 98.55, 98.65, 98.75, 98.85 or 98.95% (w/w), or fractions in between.

There are also several commercially available stabilized chlorites or diluted forms of stabilized chlorites (“stabilized chlorite solutions”), including, but not limited to, WF10, Oxovasin™, and OXO-K993. In an embodiment of the application, the stabilized chlorite is OXO-K993 and the stabilized chlorite solution is a solution comprising OXO-K993. OXO-K993 is also referred to as tetrachlorodecaoxide or TCDO. Oxovasin™ (Nuvo Manufacturing, Wanzleben, Germany), for example, is a OXO-K993 solution of particular interest in the practice of the present application. Oxovasin™ also sold under the brand name Oxoferin™, is available commercially and comprises about 2% (w/v) OXO-K993, about 2% glycerol (w/v) and about 96% water (w/v). In particular, 1 ml of Oxovasin™ comprises about 0.85 mg (or about 0.085% w/w) of chlorite in 1.0 ml water. The pH of Oxovasin™ is between 10.75 and 11.90. WF10 is a 10% (w/v) diluted aqueous solution of OXO-K993.

In an embodiment of the application, OXO-K993 is prepared using the following method:

Sodium chlorite (NaClO₂) and sodium hypochlorite (NaOCl) are mixed in a molar ratio of 4.8 to 1 in Water for Injection (WFI). The pH of the solution should be greater than pH 11.0. After addition of the catalyst, chlorylsulfuric acid [ClO₂⁺] [HSO₄⁻], to this mixture the following reaction can be observed:

2ClO₂⁻+OCl⁻+2H⁺→2ClO₂+Cl⁻+H₂O (1)

The pH of the solution decreases. A portion of the chlorite is oxidized to chlorine dioxide (ClO₂) in the redox process described by Equation (1). In an equilibrium reaction, the developing chlorine dioxide forms an intense brown charge-transfer complex with the excess unoxidized chlorite, as shown in Equation (2):

ClO₂+ClO₂⁻ [symbol] [Cl₂O₄]⁻ (2)

9.65 mmol (per kg of the reaction solution) of sodium carbonate peroxohydrate (2Na₂CO₃.3H₂O₂) is then added to the solution. Upon addition of sodium carbonate peroxohydrate, part of the chlorine dioxide is reduced back to chlorite, and oxygen is formed simultaneously:

2ClO₂+H₂O₂+2OH⁻→2ClO₂⁻+O₂+2H₂O (3)

After a suitable time, for example 15 minutes, 102 mmol (per kg of the reaction solution) of sodium peroxide (Na₂O₂) is added to the solution, which becomes completely decolorized as the remaining chlorine dioxide is reduced completely to chlorite. From sodium peroxide, oxygen evolves in a slow process that typically requires at least 4 weeks (Equation 4). Simultaneously, hydroxyl ions are formed, resulting in a high pH value (pH>13) of the solution, which thereby stabilizes the active substance chlorite.

2O₂²⁻+2H₂O→O₂+4OH⁻ (4)

The final reaction product, OXO-K993, resulting from this synthesis is a stable aqueous solution, which contains the active substance, chlorite (4.25%), together with the anions chloride (2.0%), chlorate (1.5%), and sulfate (0.7%), and sodium as the cation.

The skilled artisan will recognize that any chemically stabilized chlorite solution, including derivatives of OXO-K993, WF10, Oxovasin™ or other chlorite-based solutions and their derivatives, are well within the scope of the application. These solutions can be used in the compositions, methods and uses of the present application and, as such, the scope of the application is not necessarily limited to use of the products described herein.

In an embodiment of the application, the stabilized chlorite is present in the composition in an amount of 0.5, 0.75, 1.0, 1.25, 1.5, 1.75, 2.0, 2.25, 2.5, 2.75, or 3% (w/w), or fractions in between.

In a further embodiment of the present application, the foamable composition comprises about 0.01% (w/w) to about 1.0% (w/w), about 0.02% (w/w) to about 0.5% (w/w), about 0.05% (w/w) to about 0.20% (w/w) or about 0.08% (w/w) to about 0.1% (w/w) of chlorite ion (ClO₂⁻).

The pH of the stabilized chlorite solution is greater than about 8, greater than about 9, or greater than about 10. In an embodiment the pH of the stabilized chlorite solution is about 8 to about 13, about 9 to about 12.5, or about 10 to about 12.

The chlorite for use in the stabilized chlorite of the present application may be obtained from any available source and is commercially available. In an embodiment, the chlorite is sodium chlorite, although a person skilled in the art would appreciate that other metal salts of chlorite can be used.

### B. Foaming Agents and Foam Stabilizers

The foaming agents of the present application are operable to form a foam with the stabilized chlorite solution without: (1) substantially affecting the pH of the composition and (2) decreasing the stability of the composition to the extent that it is no longer commercially viable. Furthermore, in an embodiment, the foaming agents are operable without requiring the use of one or more additional agents that increase the skin irritancy of the composition.

In an embodiment of the application, the foaming agents and foam stabilizers are any polymer, surfactant, protein or other material known to promote the formation of a foam. In embodiments of the application, the foaming agent and foam stabilizer are selected from poloxamer copolymers, polyvinylalcohol (PVA), polyvinylpyrrolidone (PVP), polysorbates (Tween™), cellulose ethers and coco-derived amphoteric surfactants and combinations thereof.

In one embodiment, the PVA is a highly hydrolyzed form of PVA. The foamable compositions of the application may therefore comprise PVA that is at least about 95%, 96%, 97%, 98%, 99% or 100% hydrolyzed. In another embodiment, the PVA is less hydrolyzed and the foamable compositions comprise PVA that is at least about 50%, 60%, 70%, 80% or 90% hydrolyzed. In yet another embodiment, the foamable compositions comprise PVA that is about 80 to about 85% or about 85 to about 99% hydrolyzed. In an embodiment, the more hydrolyzed PVA is used when the stabilized chlorite is a diluted aqueous solution of OXO-K993.

In one embodiment of the application, the poloxamer copolymer is poloxamer 188, poloxamer 407, poloxamer 124, poloxamer 338 or poloxamer 237, or a mixture thereof. In another embodiment the poloxamer copolymer is poloxamer 188, poloxamer 407 or poloxamer 124, or a mixture thereof.

In one embodiment of the application, the polysorbate is polyoxyethylene (20) sorbitan monolaurate (Tween™ 20), polyoxyethylene (20) sorbitan monopalmitate (Tween™ 40), polyoxyethylene (20) sorbitan monostearate (Tween™ 60) or polyoxyethylene (20) sorbitan monooleate (Tween™ 80) or a mixture thereof. In another embodiment, the polysorbate is polyoxyethylene (20) sorbitan monolaurate. In another embodiment, the polysorbate is polyoxyethylene (20) sorbitan monopalmitate.

In one embodiment of the application, the cellulose ether is hydroxypropycellulose (HPC), methyl cellulose, hydroxyethylcellulose (HEC), hydroxypropylmethylcellulose (HPMC), hydroxyethylmethylcellulose (HEMC), or carboxymethylcellulose sodium or a mixture thereof. In another embodiment, the cellulose ether is HPC. In another embodiment, the cellulose ether is Methocel™.

In one embodiment of the application, the coco-derived amphoteric surfactant is sodium cocoamphoacetate, sodium cocoamphopropionate, disodium cocoamphodiacetate or disodium cocoamphodipropionate, or a mixture thereof. In another embodiment, the coco-derived amphoteric surfactant is disodium cocoamphodiacetate.

It has surprisingly been found that certain combinations of foaming agents and, optionally, foam stabilizers produce foams with enhanced foam quality and stability. In an embodiment of the application, the foamable compositions include those wherein the foaming agent and, optionally, foam stabilizer comprises, consists or consists essentially of a poloxamer, a cellulose ether and, optionally, PVA. In another embodiment, the foamable compositions include those wherein the foaming agent, and optionally, foam stabilizer comprises, consists or consists essentially of a poloxamer, a cellulose ether and PVA. In another embodiment, the foamable compositions include those wherein the foaming agent, and optionally, foam stabilizer comprises, consists or consists essentially of a poloxamer and PVA. In another embodiment, the foamable compositions include those wherein the foaming agent, and optionally, foam stabilizer comprises, consists or consists essentially of a cellulose ether and PVA. In a further embodiment, the foamable compositions include those wherein the foaming agent, and optionally, foam stabilizer comprises, consists or consists essentially of a cellulose ether and polysorbate. In a further embodiment, the foamable compositions include those wherein the foaming agent, and optionally, foam stabilizer comprises, consists or consists essentially of a poloxamer. In a further embodiment, the foamable compositions include those wherein the foaming agent, and optionally, foam stabilizer comprises, consists or consists essentially of a cellulose ether. In a further embodiment, the foamable compositions include those wherein the foaming agent, and optionally, foam stabilizer comprises, consists or consists essentially of PVA.

When the foaming agent, and optionally, foam stabilizer is a mixture of a poloxamer and HPC, it is an embodiment that the ratio of poloxamer:HPC is about 1:1 to about 1:2. When the foaming agent, and optionally, foam stabilizer is a mixture of a poloxamer, HPC and PVA, it is an embodiment that the ratio of poloxamer:HPC:PVA is about 1:1:2 to about 1:1:6.

In another embodiment of the application, the at least one foaming agent, and optionally, foam stabilizer is present in the composition in an amount of 1.15, 1.25, 1.35, 1.45, 1.55, 1.65, 1.75, 1.85, 1.95, 2.05, 2.15, 2.25, 2.35, 2.45, 2.55, 2.65 or 2.75% (w/w), or fractions in between.

Without wishing to be bound by theory, the poloxamer copolymers, polyvinylalcohol (PVA), polyvinylpyrrolidone (PVP), polysorbates (Tween™), and coco-derived amphoteric surfactants of the application are considered foaming agents while the cellulose ethers of the application are considered foam stabilizers.

### C. Water

The foamable composition further comprises water. The water may be a constituent of the stabilized chlorite solution or may be combined with a stabilized chlorite. In one embodiment, about 97.5% to about 98.925% (w/w) of stabilized chlorite and water (i.e. the stabilized chlorite solution) is present in the composition. In another embodiment, about 85% to about 98.5% (w/w) water is present when the composition comprises about 0.5% to about 3% (w/w) of stabilized chlorite. In an embodiment, the water is pharmaceutical grade water, such as distilled and/or deionized water or water-for-injection.

### D. Buffering Agent

In an embodiment of the application, the foamable composition further comprises a buffering agent. In an embodiment, the buffering agent is an inorganic base or an organic base or salt thereof, such as an alkali metal base or an amine or amide, or a salt thereof. In one embodiment, the alkali metal base is sodium carbonate, pentasodium triphosphate, potassium pyrophosphate, sodium pyrophosphate, sodium citrate, potassium citrate, potassium carbonate, sodium bicarbonate or potassium bicarbonate, or a mixture thereof. In another embodiment, the alkali metal base comprises, consists of or consists essentially of sodium carbonate. In a further embodiment, the inorganic base is present in the foamable compositions of the application in an amount of about 0.075 wt %.

In a further embodiment of the application, the buffering agent is present in the composition in an amount of 0.005, 0.015, 0.025, 0.035, 0.045, 0.055, 0.065, 0.075, 0.085 or 0.095% (w/w), or fractions in between.

### E. Propellants

The compositions of the application are foamable by manual aeration. Accordingly, in one embodiment the formulation is propellant-free. However, the addition of propellants to inherently foamable formulations (e.g. those formulations foamable by manual aeration) may provide a more consistent delivery of the active agent. For example, addition of a propellant to a foamable formulation may be useful in producing metered dosing of the composition, as required by certain regulatory bodies to prevent over- or under-dosing. The addition of a propellant however is not key for deriving a foam from the compositions of the application.

It is therefore an optional embodiment for the compositions of the present application to include a propellant. In one embodiment, the propellant is from about 3 to about 45% (w/w) of the foamable composition. In another embodiment, the propellant is about 5%, about 10%, about 15%, about 20%, about 25%, about 30%, about 35%, about 40%, or about 45% (w/w) of the foamable composition.

In some aspects, the compositions are capable of being formulated into an aerosol foam or a mousse by addition of propellant to the composition. The propellant may form a separate layer on the composition or the propellant may be emulsified or miscible in the composition. The use of propellants in a foamable composition is discussed in U.S. Pat. No. 7,651,990, the contents of which are incorporated by reference in their entirety.

In embodiments of the application, suitable propellants are chosen from chlorofluorocarbons (CFCs), hydrochlorofluorocarbons (HCFCs), hydrofluorocarbons (HFCs), perfluorinated alkanes, and lower alkanes (C₁-C₅) as well as nitrous oxide dimethyl ether and other solvent-soluble propellants. In an embodiment, suitable lower alkanes include propane, butane, and isobutane. In other embodiments, the propellant comprises a 70/30 mixture of propane/isobutane. In other embodiments, the propellant is A-46, which is a blend of 84% A-31 isobutane and 16% A108 propane.

In order to produce an aerosol composition, the composition is first formulated and charged into an appropriate pressure-rated container. A suitable propellant may then be added to the composition under pressure at about 1-30%, or about 3-20%, by volume. Non-limiting examples of canisters useful in dispensing propellant-based foams include Aptar Pharma's Bag on Valve (BOV) and other suitable Aptar systems (Aptar Pharma, France).

In one embodiment, the foamable composition of the application is mixed with a propellant in a ratio from about 70:30 to 99:1 (% w/w). In another embodiment, the foamable composition of the application is mixed with a propellant in a ratio from about 85:15 to 97:3 (% w/w). In a further embodiment, the foamable composition of the application is mixed with a propellant in a ratio of about 90:10 (% w/w). In an embodiment, the propellant is A-46.

In another embodiment, the process for preparing a propellant formulation comprises adding the propellant (e.g., at about 10% concentration by weight) to the foamable composition (e.g., at about 90% concentration by weight) and pressure-filling the cans, for example, using a Kartridg Pak pressure filler (available, for example, from Oystar, North America).

### F. Other Components

In embodiments of the present application, the compositions further comprise other additives or agents that are desired for particular applications. Such additives or agents include, but are not limited to, humectants, solvents, antibiotics, dyes, perfumes, fragrances and the like. In one embodiment, the compositions comprise an anti-oxidant. In an embodiment, the anti-oxidants for use in the present application include butylated hydroxytoluene, butylated hydroxyanisole, ascorbyl linoleate, ascorbyl dipalmitate, ascorbyl tocopherol maleate, calcium ascorbate, carotenoids, kojic acid and its pharmaceutically acceptable salts, thioglycolic acid and its pharmaceutically acceptable salts (e.g., ammonium), tocopherol, tocopherol acetate, tocophereth-5, tocophereth-12, tocophereth-18, or tocophereth-80, or mixtures thereof.

### G. Foam Characteristics

Compositions of the present application produce foams with measurable characteristics. In certain aspects, qualities such as foam stability, easiness to spread and appropriate breakability upon application to the skin or wound are desirable features. These characteristics can be measured by conducting foam formation and foam collapsibility experiments. Foam formation (foam height vs time), for example, is predictive of the generation of a sprayable/spreadable foam. The rate of collapsibility is an important property in the appropriate administration of the foam.

The height of the water-based foams of the present application can remain stable (in terms of foam height) up to 24 hr or longer, although foam cells may collapse to form larger cells. This is unlike other products that have been tested, which after only a few hours have no discernable foam.

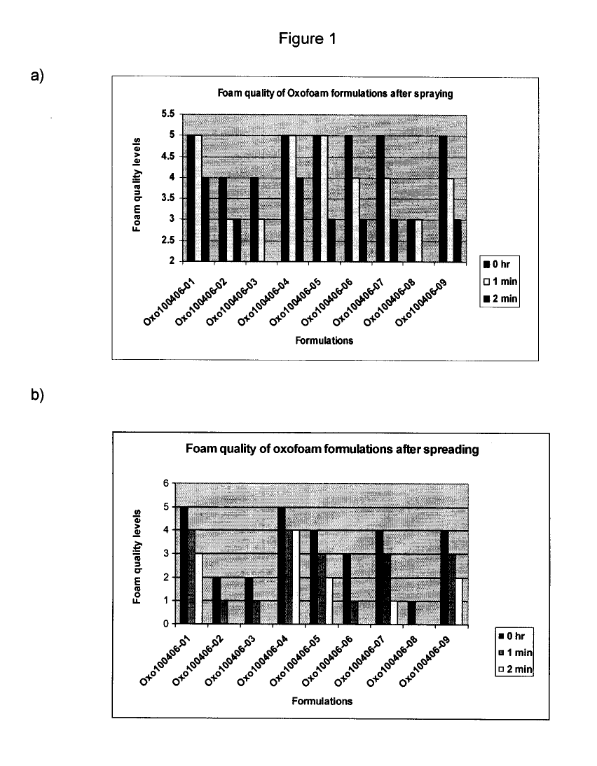

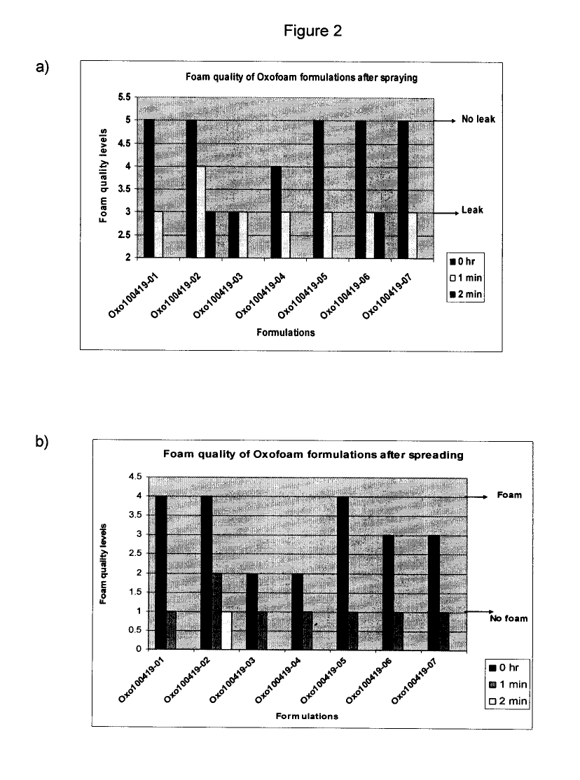

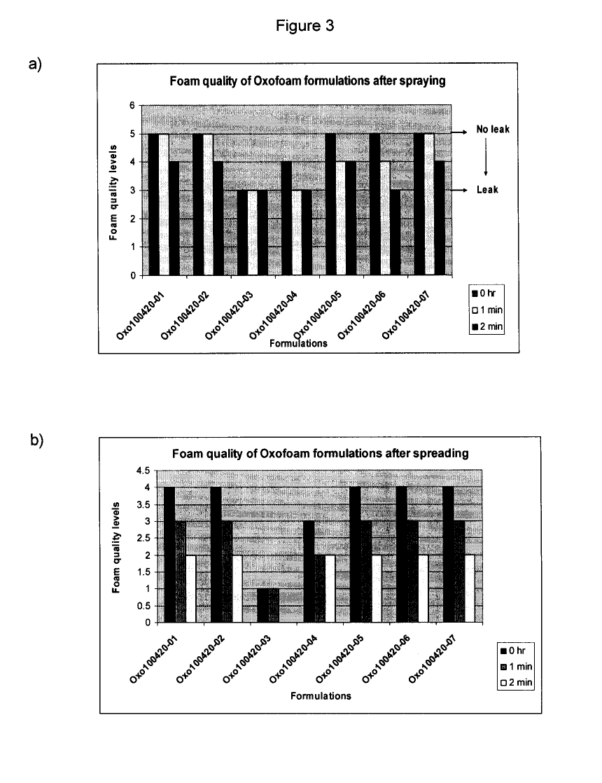

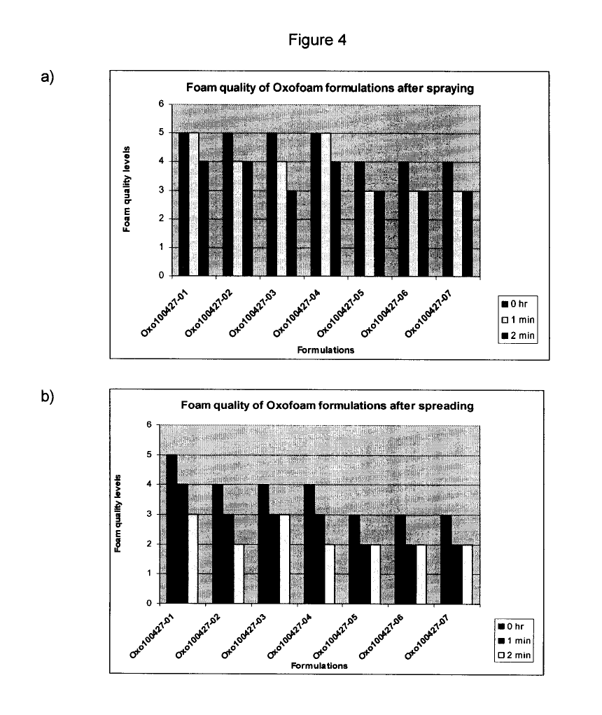

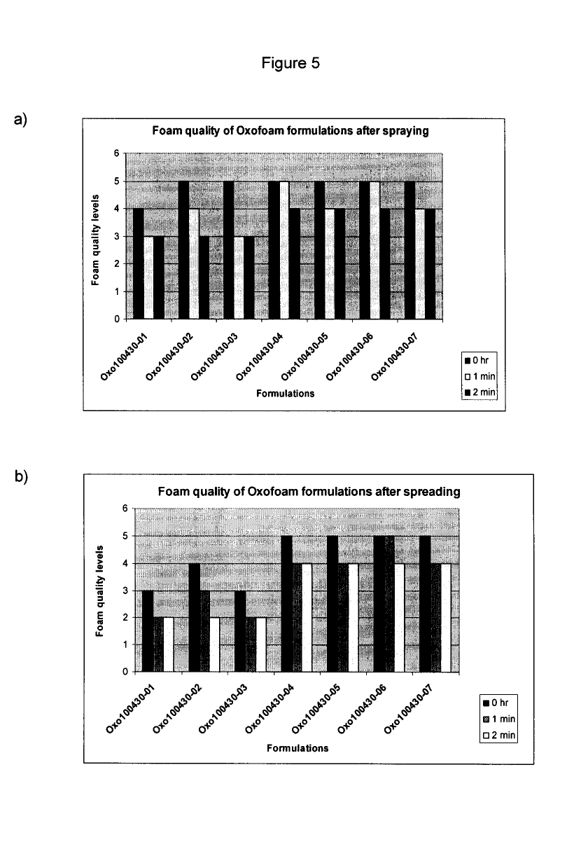

With reference to the figures, foam quality may be monitored by measuring foam leakiness and consistency (following spraying or spreading, respectively) up to 2 minutes after application (see, for example, FIGS. 1 to 5). Foam qualities, such as volume, density and surface tension can also be measured.

In certain embodiments of the application, the foamable compositions have the advantage of maintaining chemical or physical stability over a period of time. In Table 10, for instance, the pH of certain formulations were monitored over the course of 1 week.

In certain embodiments of the invention, the foamable compositions are substantially stable with respect to chemical or physical attributes over a predetermined period of time. The measurable attributes may include, but are not limited to, percentage of active, percentage of impurities, pH, or visual attributes, such as colour and the presence of particulates. In other embodiments, the composition is substantially stable following storage for about 1, 2, 3, 4, 8, 9, 10, 12, 16, 20 or 24 weeks at room temperature (RT).

In an embodiment of the application, the foamable composition when foamed does not collapse to a liquid phase for at least about 30 seconds at 37° C. or at skin temperature. In a further embodiment, the composition when foamed does not collapse to a liquid phase for at least about 60 seconds at 37° C. or at skin temperature. In yet another embodiment, the composition when foamed does not collapse to a liquid phase for at least about two minutes at 37° C. or at skin temperature. In a further embodiment, the composition when foamed does not collapse to a liquid phase for at least about five minutes at 37° C. or at skin temperature.

In further embodiments of the application, the unfoamed composition has a pH of about 5 to about 12. In one embodiment, the pH of the composition ranges from about 5 to about 6.5, about 6.5 to about 8, about 8 to about 9.5, or about 9.5 to about 12. In another embodiment, the pH of the composition is basic and ranges from about 10 to about 12.5, about 10 to about 11.5, or about 10 to about 10.5. In another embodiment, the foamable composition has a pH of 10 or above.

To maintain its pH, the composition may include a buffering agent, as described herein, or a suitable alternative. In one embodiment, the foamable compositions have the advantage of maintaining pH over a period of one, two or three months in storage. The foamed composition may adjust its pH to that of the skin once applied.

### H. Specific Compositions of the Application

In embodiments of the application there is included a foamable composition comprising:

about 97.5% to about 98.925% (w/w) of stabilized chlorite and water;

about 1% to 2.5% (w/w) of a foaming agent and, optionally, a foam stabilizer, selected from a poloxamer, HPC and, optionally, PVA, and mixtures thereof; and

about 0.05% to about 0.1% (w/w) of an inorganic base.

When the foaming agent and, optionally, foam stabilizer, is a mixture of a poloxamer and HPC, it is an embodiment that the ratio of poloxamer:HPC is 1:1 to 1:2. When the foaming agent and, optionally, foam stabilizer is a mixture of a poloxamer, HPC and PVA, it is an embodiment that the ratio of poloxamer:HPC:PVA is 1:1:2 to 1:1:6.

In a further embodiment of the present application there is included a foamable composition comprising:

about 97.5% to about 98.925% (w/w) of stabilized chlorite and water;

about 0.5% (w/v) poloxamer 407;

about 0.5% (w/v) HPC HY117;

about 0.925% (w/v) PVA; and

about 0.075% (w/v) of sodium carbonate.

In a further embodiment of the present application there is included a foamable composition comprising:

(i) about 1% to about 3% (w/w) stabilized chlorite;

(ii) about 95% to about 97% (w/w) water;

(iii) about 0.5% (w/w) poloxamer 407;

(iv) about 1% to about 2% highly hydrolyzed PVA (e.g. 99% hydrolyzed PVA); and

(v) about 0.08% to about 0.1% sodium carbonate.

In a further embodiment of the present application there is included a foamable composition comprising:

(i) about 1% to about 3% (w/w) stabilized chlorite;

(ii) about 95% to about 97% (w/w) water;

(iii) about 0.25% (w/w) poloxamer 407;

(iv) about 0.25% (w/w) methyl cellulose;

(iv) about 1% to about 2% highly hydrolyzed PVA (e.g. 99% hydrolyzed PVA); and

(v) about 0.08% to about 0.1% sodium carbonate.

### I. Foams

The present application also includes a foam comprising a foamable composition of the application.

In still a further embodiment of the present application, there is provided a foam comprising:

(i) an aqueous stabilized chlorite solution;

(ii) at least one foaming agent; and, optionally,

(iii) a buffering agent.

In yet a further embodiment of the present application, there is provided a foam comprising:

(i) stabilized chlorite;

(ii) at least one foaming agent;

(iii) water, and, optionally,

(iv) a buffering agent.

In other embodiments, the foams of the application further comprise at least one foam stabilizer.

### III. Methods and Uses of the Application

The compositions of the present application are novel, therefore the application further includes all uses of these compositions as well as methods which include these compositions. In a particular embodiment, there is included a use of the compositions of the present application as a medicament or antimicrobial agent.

Advantageously, the compositions of the present applicant are useful in topical or transdermal medicaments. The composition of stabilized chlorite solutions as foams allows a more targeted and clean administration of the active agent (chlorite) to a desired location.

The compositions are particularly useful for the treatment of a condition for which topical or transdermal administration of a stabilized chlorite solution is beneficial. Therefore the present application also includes a method for treating a condition comprising applying an effective amount of a foamable composition or a foam of the application to a subject in need thereof. In one embodiment, the foamable composition or foam is applied to the skin of the subject alone or in combination with a bandage. In another embodiment, the foamable composition or foam is applied to a cavity of the subject alone, or in combination with a bandage.

Examples of conditions that may be treated with the compositions of the application include, but are not limited to skin diseases or disorders, such as topical or neuro dermatitis, psoriasis, herpes simplex, herpes zoster and acne, for infections or burns, for wound healing, including pressure, post-operative and post-traumatic wound healing, as well as chronic wound healing in the case of diabetic ulcers, venous ulcers, arterial ulcers, decubitus ulcers and the like.

The present application also includes a method for increasing macrophage and/or phagocyte stimulation comprising administering an effective amount of a foamable composition or a foam of the application to a subject in need thereof.

Also included in the present application is a use of a foamable composition or foam of the application for wound healing.

Further included in the present application is a use of a foamable composition or foam of the application as an antimicrobial agent.

Further included in the present application is a use of a foamable composition or foam of the application for increasing macrophage and/or phagocyte stimulation.

In one embodiment, the treatment is administered once a day. In another embodiment, the treatment is administered twice a day. In still another embodiment, the treatment is administered three times a day. In yet another embodiment, the treatment is administered four times a day. In a further embodiment, the treatment is administered one to two times a day for one, two, three, four, five, six or seven days. In still a further embodiment, the treatment is administered at least once a day for a longer term such as 1, 2, 3, 4, 5, 6, 7, 8, 9, 10, 11 or 12 weeks. In an even further embodiment, the treatment is administered at least once a day until the condition has ameliorated to where further treatment is not necessary.

In another embodiment, the treatment is administered at least once per week. In another embodiment, the treatment is administered twice per week. In still another embodiment, the treatment is administered three times per week. In yet another embodiment, the treatment is administered four times per week. In yet another embodiment, the treatment is administered five times per week. In yet another embodiment, the treatment is administered six times per week. In a further embodiment, the treatment is administered one to six times per week for one, two, three, four, five, six or seven weeks. In still a further embodiment, the treatment is administered at least once per week for a longer term such as 1, 2, 3, 4, 5, 6, 7, 8, 9, 10, 11 or 12 weeks. In an even further embodiment, the treatment is administered at least once per week until the condition has ameliorated to where further treatment is not necessary.

In other embodiments of the application the foamable compositions are, if desired, presented in a canister, foaming dispenser, or other closure system approved by the Food and Drug Administration (FDA) or other regulatory body, which contain one or more unit dosage forms containing the active solution. In an embodiment, the canister or dispenser is also accompanied by a notice associated with the container in a form prescribed by a governmental agency regulating the manufacture, use, or sale of pharmaceuticals, the notice indicating approval by the agency.

Compositions of the present application are useful and effective when applied topically to treat a condition. The amount of the active agent present in the composition will be the amount that is therapeutically effective, i.e., an amount that will result in the effective treatment of the condition (e.g., wound healing) when applied. The therapeutically effective amount will vary depending on the subject and the severity of the affliction and can be determined routinely by one of ordinary skill in the art. Exemplary dosing of a chlorite-containing solution for the treatment of ulcerative wounds, for example, is provided in Hinz et al., The Lancet (1986), the contents of which is incorporated by reference in its entirety.

### IV. Dispensing System

In embodiments, the compositions of the present application are formulated into products that can be dispensed from a reservoir using a release assembly (e.g., a hand pump) to dispense an amount of the composition whenever the release assembly is put into action. The amount of the composition dispensed by the pump may or may not be metered to dispense a consistent amount of composition.

Accordingly, the application also includes a dispenser comprising a reservoir operably linked (e.g., in fluid communication) with a release assembly, wherein the reservoir contains a foamable composition of the application, and wherein the release assembly allows the foamable composition to be released as a foam. In an embodiment, the release assembly is a dispensing head.

The application also includes a pressurized container, the container holding a foamable composition of the present application and optionally a propellant. In an embodiment, a release assembly comprising a nozzle or sprayer is operably linked to the container (e.g., a release assembly for dispensing a foam from an aerosol spray can wherein the release assembly is in fluid communication with the spray can), wherein the release assembly allows the foamable composition and optionally a propellant to be released as a foam.

Non-limiting examples of pumps useful in dispensing the compositions of the application include the Rexam M3, G3 and F3 pump types foaming head (Rexam PLC, London, England) and the Meadwestvaco Ocean T and Ocean H spray heads (Meadwestvaco Corp. Virginia, USA). In preliminary foaming trials, a Rexam M3 S-10 white pumps PE (polyethylene) having a 50 ml reservoir was used. However, the compositions of the application are not limited to being dispensed from only one type of dispenser or through only one type of hand pump. Further, the dispenser or pump head may include additional or altered features that assist in optimizing foam stability. These features include, but are not limited to, the inclusion of meshes in the pump head and varied dip tube and nozzle lengths.

The compositions of the application are suitable for topical use including administration to infected or inflamed skin, abraded skin or open wounds. In further embodiments, non-aerosol compositions of the application are contained in a non-aerosol dispenser equipped with a conventional hand pump, and the compositions are pumped onto the hands or other areas of the body. The pumping action required to dispense the compositions will create a discrete volume of a dispensed composition as a stable foam.

The following non-limiting examples are illustrative of the present application:

### V. EXAMPLES

### Materials

The source for the materials used in the compositions described below was as follows:

Disodium Cocoamphodiacetate (DCAM): Rhodia UPOD12_{X}06. Poloxamer 188 (Pluronic F68): Hyclone 070530191F. Poloxamer 407: Spectrum QU2503. Polysorbate 20 (Tween™ 20): Spectrum YD1021. Polysorbate 40 (Tween™ 40): Spectrum XJ0977. Polysorbate 80 (Tween™ 80): JT Baker C19626. Polyvinyl alcohol 80% hydrolyzed (PVA 80%): Sigma Aldrich MKBD2298. Polyvinyl alcohol 99% hydrolyzed (PVA 99%): Spectrum XE0797. Polyvinylpyrrolidone 30 (PVP 30): Spectrum XQ0602. Carboxymethylcellulose sodium (CMCNa): Spectrum VK0399. Hydroxyethyl cellulose (Natrosol): Hercules C02278. Hydroxypropyl cellulose HY117 (HPC HY117): Spectrum ZM3082. Methylcellulose (Methocel): Spectrum YC0112. Sodium Carbonate: Spectrum 86156. OXO-K993 and Oxovasin™ were obtained from Nuvo Manufacturing GmbH (Wanzleben, Germany).

The term “PVA” or “polyvinyl alcohol” when used in the examples without further designation refers to polyvinyl alcohol 80% hydrolyzed.

### Methods

(1) Foam Quality after Spraying and Spreading

Various container closure systems were tested during the course of the development program including a recycled soap dispenser which was used in early development work as well as the Ocean™ H Foamer from MEADWESTVACO (Richmond, Va.) and the M3-S10 Airspray® pump foamer from Rexam (London, UK).

To measure foam quality after spraying and quality after spreading, the test formulation was dispensed from a Rexam M3 S-10 white pump PE (polyethylene) having a 50 ml reservoir. The dispenser was primed twice, following which the foam was dispensed on a skin surface.

Foam quality after spraying was assessed immediately after spraying by observing whether fluid leaked from the foam (appearance of liquid at the base of the foam) and was ranked on the following three point integer scale ranging from 3 to 5:

5=No leak;
4=Light leak; and

### 3=Leak.

Foam quality after spraying was assessed with a single replicate. Foam quality after spreading was assessed by making a single sweep across freshly dispensed foam with a finger or spatula and ranking the resulting material on a five point integer scale ranging from 1 to 5 as follows:

### 5=Foam;

4=Light foam;
3=Lighter foam;
2=Very light foam; and
1=No foam.

A single replicate was also used when measuring foam quality after spreading.

### (2) Stability Tests

Generally, stability testing was conducted with 3 samples at each time point. Samples were stored in tall 4 dram Teflon®-coated amber glass vials with phenolic caps. Vials were stored in the dark in a laboratory drawer at room temperature without special controls. The volume of each stability sample was approximately 10 mL. The vials were approximately half full with an air head space. Sample stability was analyzed using a combination of techniques including the foam stability shake height test pH measurement and HPLC assay of chlorite concentration as described in the sections that follow and measured values were typically compared to those observed at t=0.

### (a) Foam Stability Height Shake Test

The vials containing the stability samples were held on a vortexer for 2 minutes. The foam stability was assessed by measuring foam height at t=0 minutes. In some cases foam heights were also measured at t=2 minutes.

### (b) pH Measurement of Foam Stability

The pH of the samples was measured using an Accumet AR15 pH meter. The chlorite ion is generally expected to be most stable in strongly alkaline environments and increases in the pH of a formulation as a function of time and can therefore be an indicator of stability problems.

### (c) Chlorite Ion Assay by HPLC of Foam Stability

The amount of chlorite in the foam was assessed using HPLC. The method uses a step-gradient anion exchange elution (for example, using an AS22 4.0×250 mm Column from Dionex) with detection at 215 nm. Eluents that may be used include a combination of 1.0-2.0 mM Na₂CO₃ and 10-25 mM NaHCO₃), or a single eluent of NaHCO₃ at a concentration of 10-50 mM.

### Example 1

### Preparation of Foamable Compositions

To prepare certain foamable compositions of the present application, polyvinyl alcohol (PVA, 80-85% hydrolyzed) was dispersed in Oxovasin™ containing previously solubilized sodium carbonate. The dispersion was then mixed until complete dissolution of PVA was achieved. This step was followed by incorporation of Poloxamer 407, or similar derivatives, and mixed until completely dissolved. A thickener as foam stabilizer such as hydroxypropyl cellulose or hydroxypropylmethyl cellulose was gradually added under agitation. Resulting mixtures were stirred and protected from light overnight.

To prepare alternate foamable compositions of the present application, polyvinyl alcohol (PVA, 99% hydrolyzed) was dispersed in purified water. The dispersion was then heated in an incubator at 90° C. until complete dissolution. After cooling to room temperature, sodium carbonate was added to the resulting solution and mixed until dissolved. This step was followed by incorporation of Poloxamer 407, or similar derivatives, and mixed until completely dissolved. Calculated amount of a 1/50 (2%) aqueous dilution of OXO-K993 concentrated solution was also added and mixed. A thickener as foam stabilizer such as hydroxypropyl cellulose or hydroxypropylmethyl cellulose was gradually added under agitation. Resulting mixtures were stirred and protected from light overnight.

Exemplary compositions prepared by the above methods are presented in Tables 1-9.

### Example 2

### Foam Quality

Foams based on the compositions of Tables 1-9 were prepared according to the following steps:

The composition was dispensed from a Rexam M3 S-10 white pump PE (polyethylene) having a 50 ml reservoir. The dispenser was primed twice, following which the foam was dispensed on a skin surface. The ability to spray or spread the foam was measured and is shown in FIGS. 1-5. In the drawings, (a) 5=no leak; 4=light leak; and 3=leak. In (b), 5=foam; 4=light foam; 3=lighter foam; 2=very light foam; and 1=no foam. Leakage and consistency were used to qualitatively assess the foam's characteristics.

### Example 3

### Foam Stability

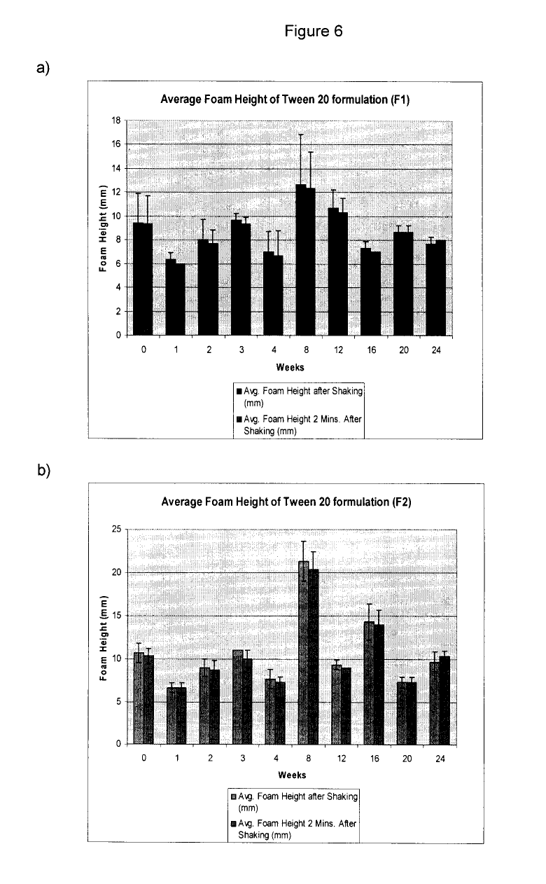

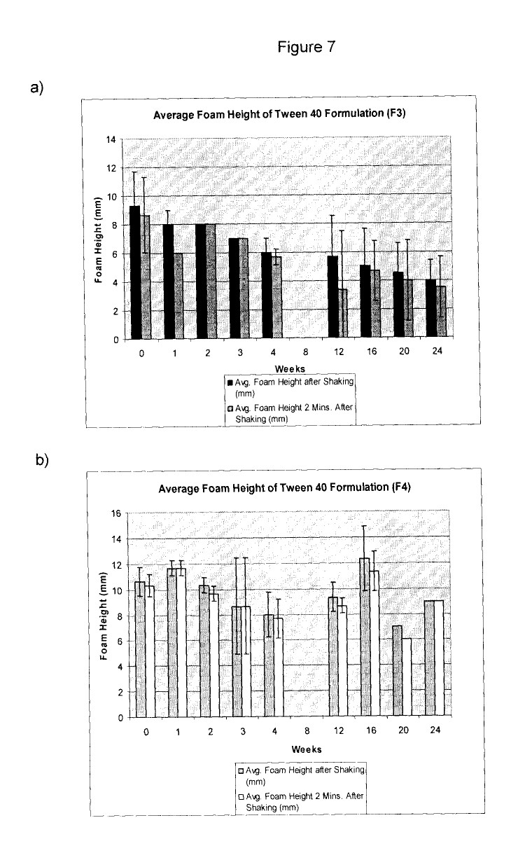

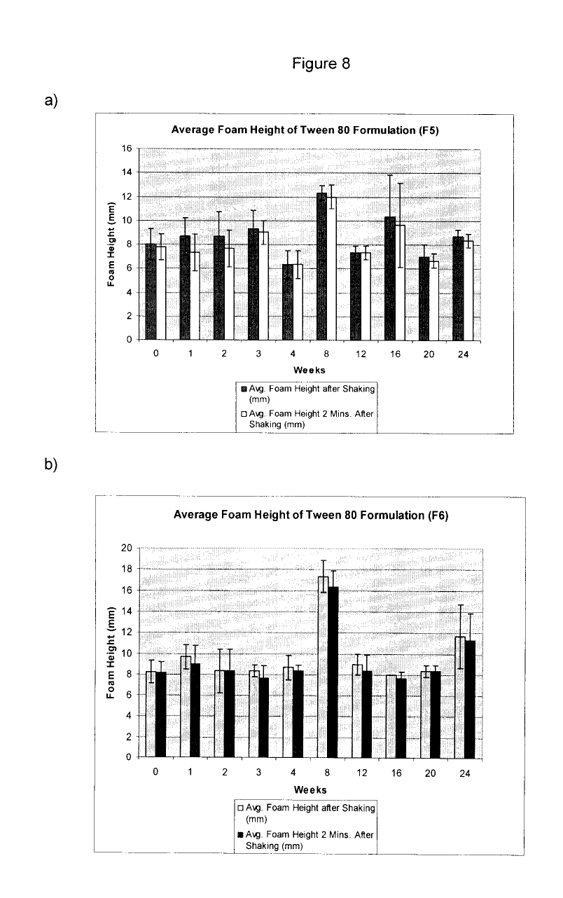

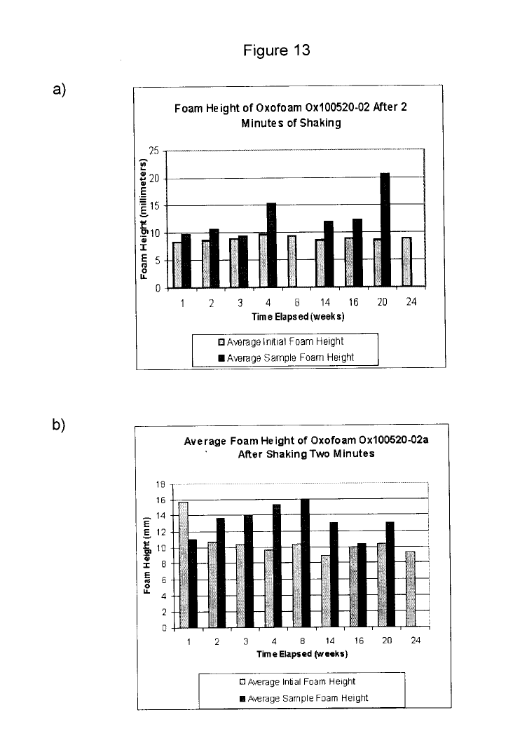

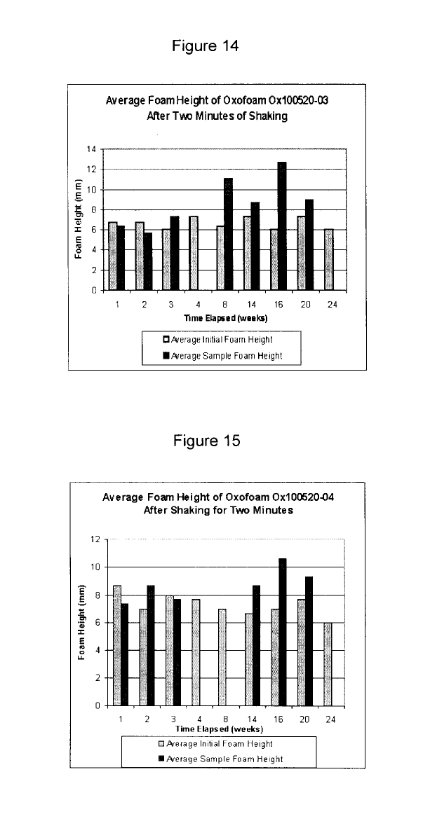

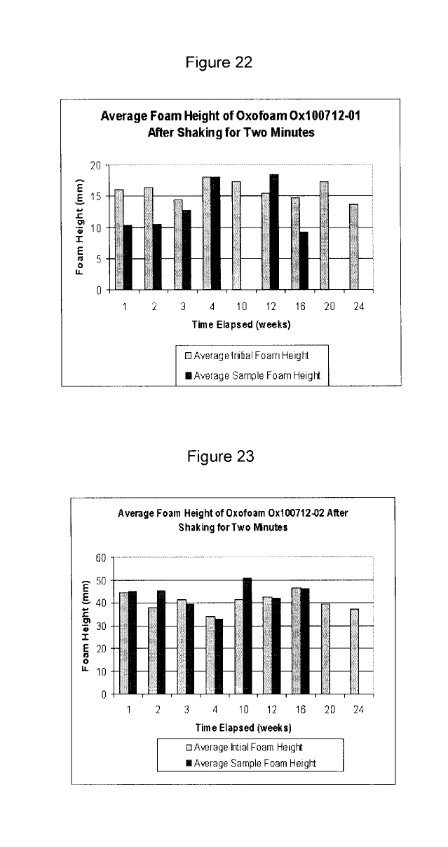

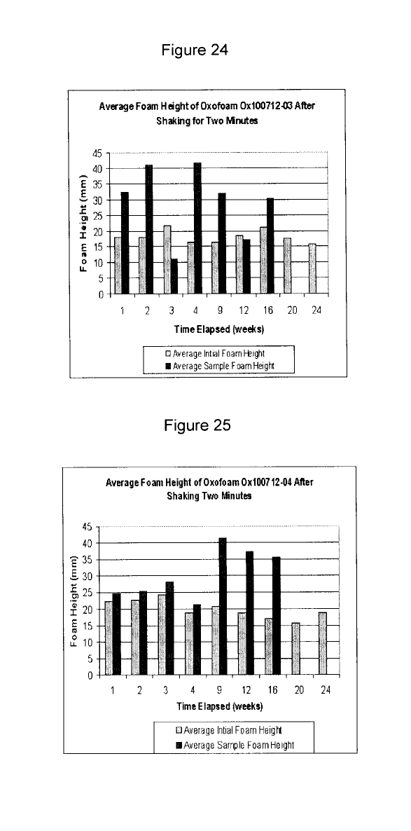

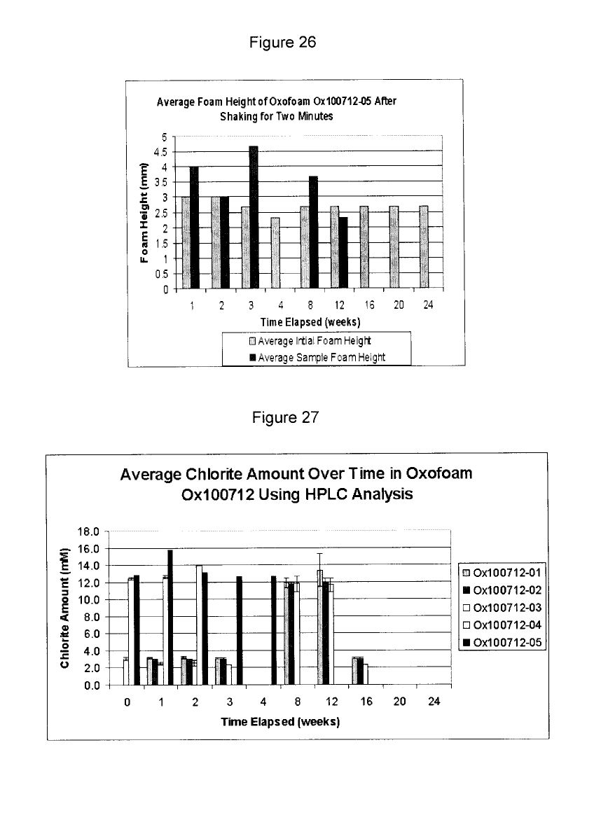

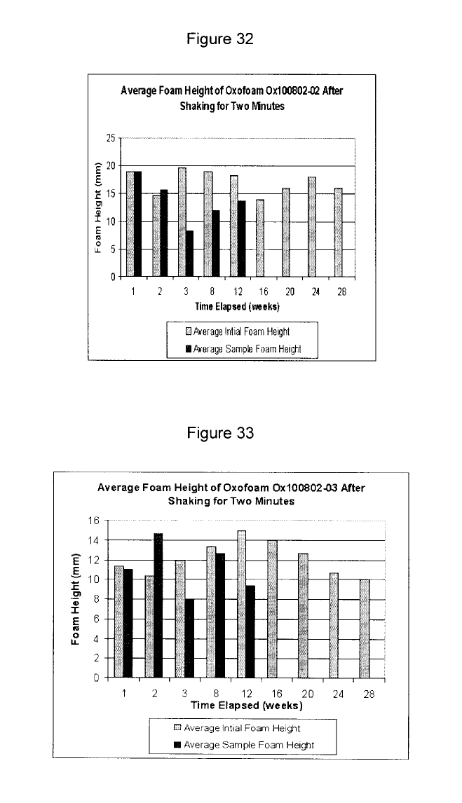

Foam stability was determined by measuring foam height at 0 and 2 minutes following manual shaking for approximately 30 seconds of samples stored for 0, 1, 2, 3, 4, 8, 12, 16, 20 and 24 weeks (FIGS. 6-8). Foam stability was determined by measuring foam height at 2 minutes following manual shaking for approximately 30 seconds of initial samples and those stored for various lengths of time (FIGS. 13-15, 22-26, and 31-33).

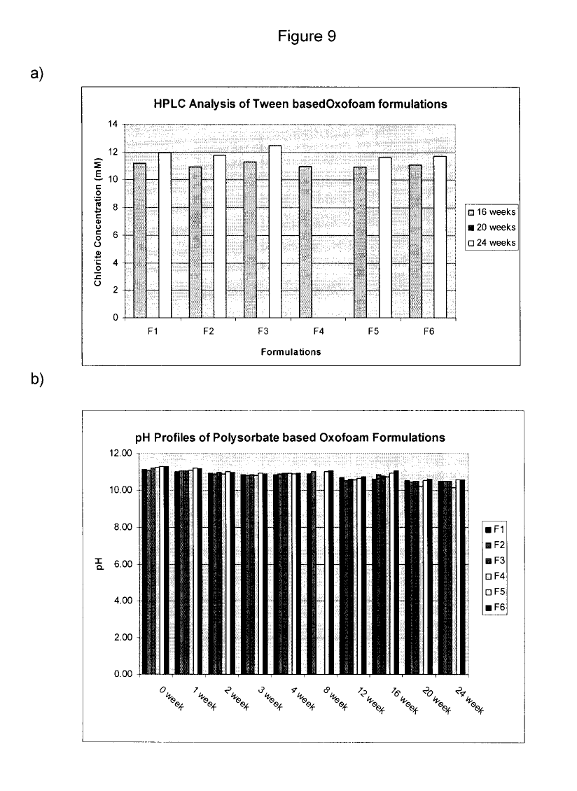

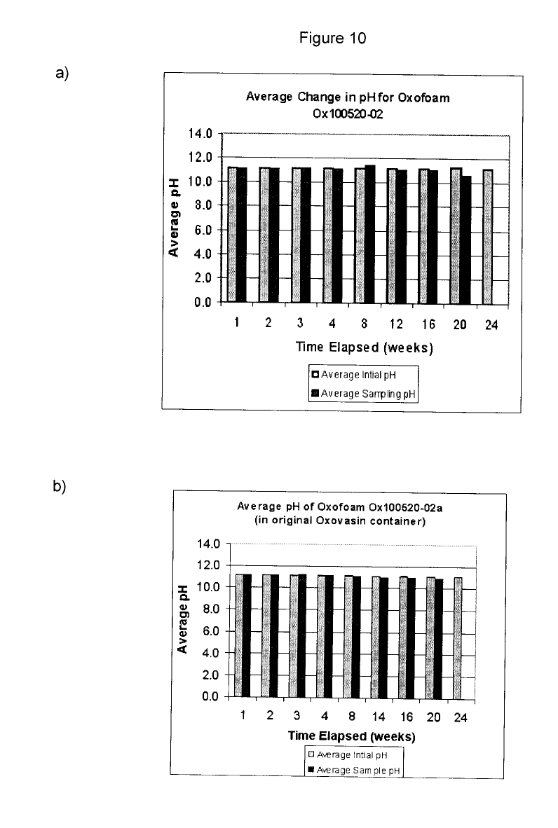

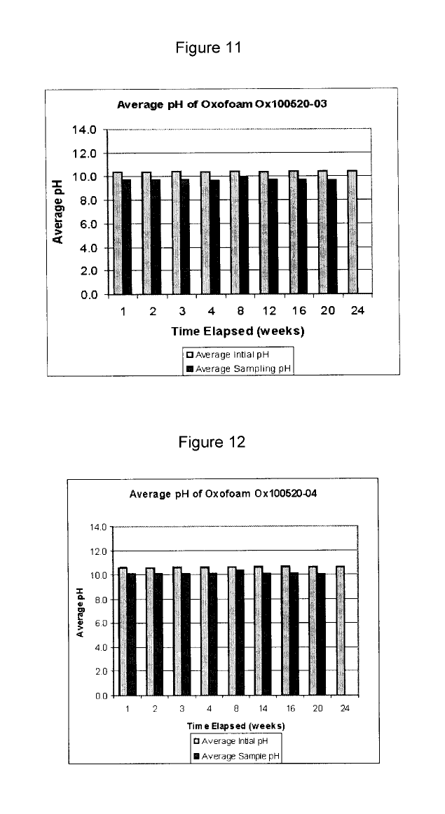

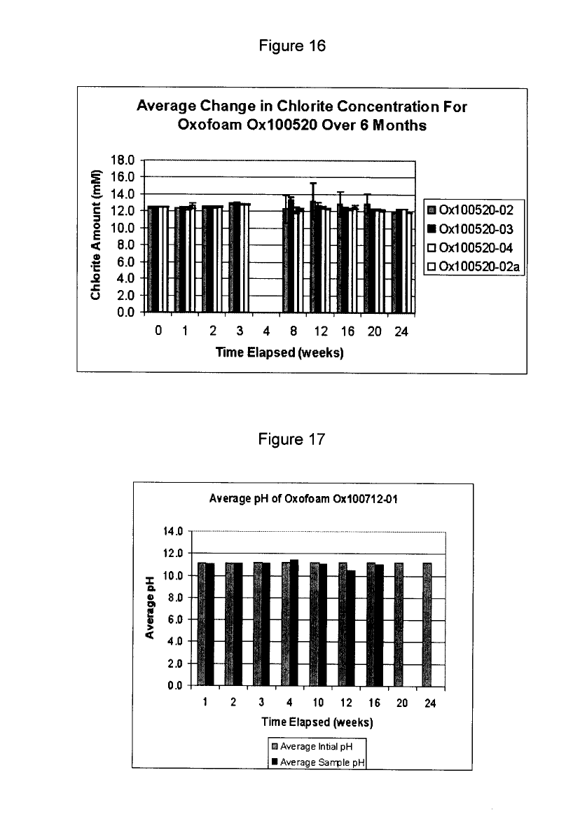

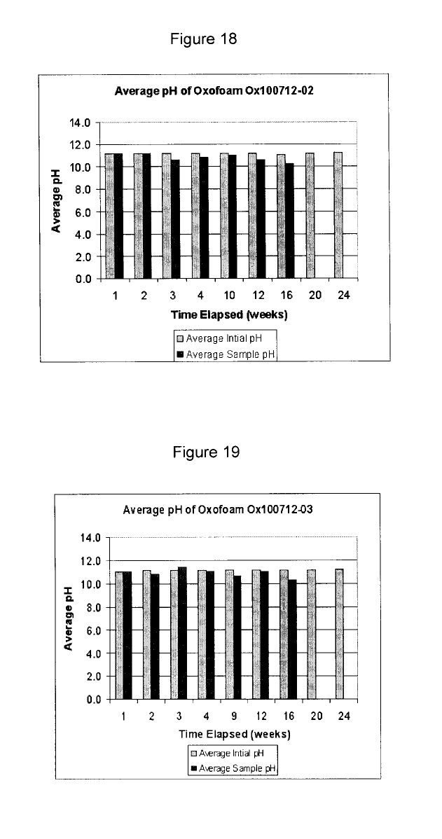

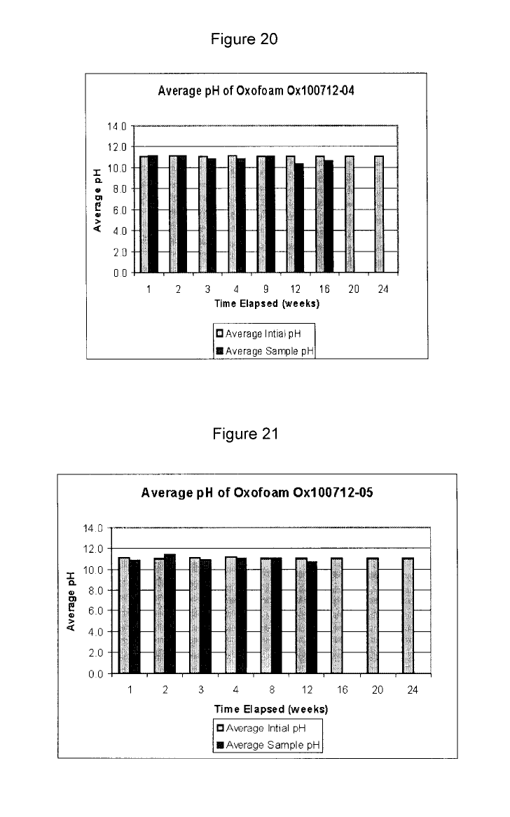

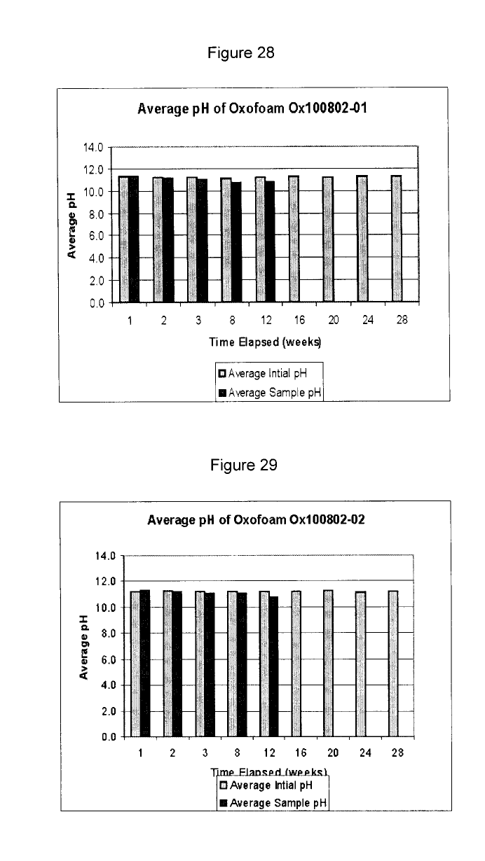

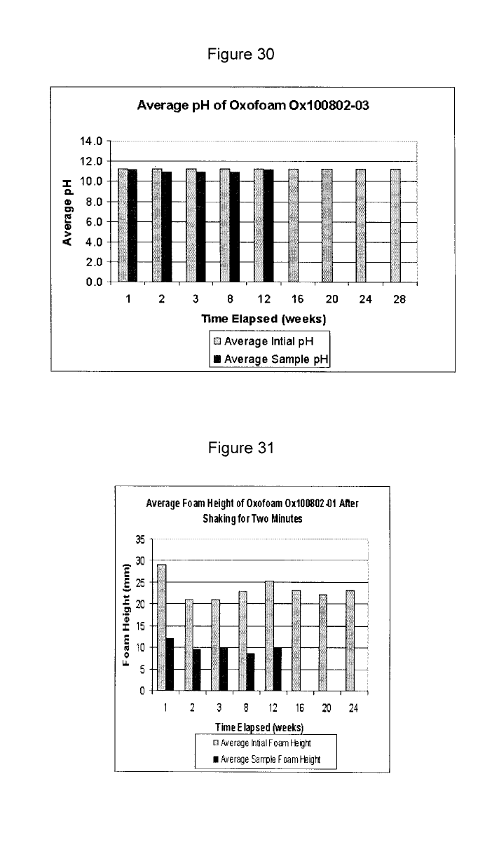

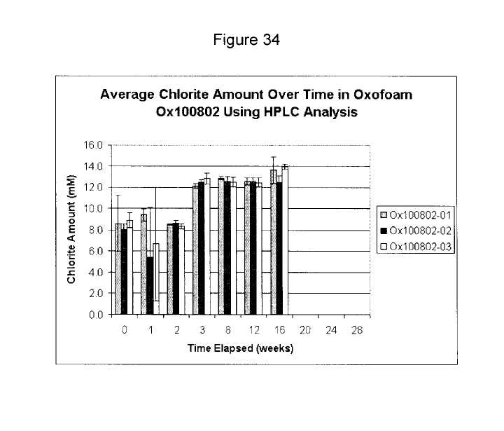

The chlorite concentration in samples stored for various lengths of times (FIGS. 9 (a), 16, 27 and 34 were analysed. Stability based on pH profiles (FIGS. 9 (b), 10-12, 17-21 and 28-30 and Table 10) of samples stored for various lengths of time were also analysed.

The relevant portions of all publications, patents and patent applications are herein incorporated by reference to the same extent as if each individual publication, patent or patent application was specifically and individually indicated to be incorporated by reference. Where a term in the present application is found to be defined differently in a document incorporated herein by reference, the definition provided herein is to serve as the definition for the term.

TABLE 1
Foamable compositions (Oxo100406-01 through Oxo100406-09)

| | Formulations | | | | | | | | |
|---|---|---|---|---|---|---|---|---|---|
| | Oxo10406- | Oxo10406- | Oxo10406- | Oxo10406- | Oxo10406- | Oxo10406- | Oxo10406- | Oxo10406- | Oxo10406- |
| | 01 | 02 | 03 | 04 | 05 | 06 | 07 | 08 | 09 |
| Ingredients | % w/w | % w/w | % w/w | % w/w | % w/w | % w/w | % w/w | % w/w | % w/w |
| Poloxamer 407 | 1.925 | | 0.5 | 0.5 | | | | | |
| Poloxamer 188 | | 1.925 | 1.425 | | | | | | |
| Polyvinyl alcohol | | | | 1.425 | | 1.425 | 1.425 | | 1.925 |
| Oxovasin | 98 | 98 | 98 | 98 | 98 | 98 | 98 | 98 | 98 |
| Pluronic F68 | | | | | 1.925 | 0.5 | | | |
| PVP-30 | | | | | | | 0.5 | 1.925 | |
| Sodium carbonate | 0.075 | 0.075 | 0.075 | 0.075 | 0.075 | 0.075 | 0.075 | 0.075 | 0.075 |

TABLE 2
Foamable compositions (Oxo100419-01 through Oxo100419-07)

| | Formulations | | | | | | |
|---|---|---|---|---|---|---|---|
| | Oxo10419- | Oxo10419- | Oxo10419- | Oxo10419- | Oxo10419- | Oxo10419- | Oxo10419- |
| | 01 | 02 | 03 | 04 | 05 | 06 | 07 |
| Ingredients | % w/w | % w/w | % w/w | % w/w | % w/w | % w/w | % w/w |
| Poloxamer 188 | 0.5 | | | 1.25 | 0.25 | 0.5 | 0.5 |
| CMCNa | | 0.5 | 1 | 0.675 | 0.25 | 0.5 | 0.5 |
| Polyvinyl alcohol | 1.425 | 1.425 | | | 1.425 | 0.925 | 1.425 |
| Oxovasin | 98 | 98 | 98.925 | 98 | 98 | 98 | 97.5 |
| Sodium carbonate | 0.075 | 0.075 | 0.075 | 0.075 | 0.075 | 0.075 | 0.075 |

TABLE 3
Foamable compositions (Oxo100420-01 through Oxo100420-07)

| | Formulations | | | | | | |
|---|---|---|---|---|---|---|---|
| | Oxo10420- | Oxo10420- | Oxo10420- | Oxo10420- | Oxo10420- | Oxo10420- | Oxo10420- |
| | 01 | 02 | 03 | 04 | 05 | 06 | 07 |
| Ingredients | % w/w | % w/w | % w/w | % w/w | % w/w | % w/w | % w/w |
| Poloxamer 407 | 0.5 | | | 1.25 | 0.25 | 0.5 | 0.5 |
| CMCNa | | 0.5 | 1 | 0.675 | 0.25 | 0.5 | 0.5 |
| Polyvinyl alcohol | 1.425 | 1.425 | | | 1.425 | 0.925 | 1.425 |
| Oxovasin | 98 | 98 | 98.925 | 98 | 98 | 98 | 97.5 |
| Sodium carbonate | 0.075 | 0.075 | 0.075 | 0.075 | 0.075 | 0.075 | 0.075 |

TABLE 4
Foamable compositions (Oxo100427-01 through Oxo100427-08)

| | Formulations | | | | | | | |
|---|---|---|---|---|---|---|---|---|
| | Oxo10427- | Oxo10427- | Oxo10427- | Oxo10427- | Oxo10427- | Oxo10427- | Oxo10427- | Oxo10427- |
| | 01 | 02 | 03 | 04 | 05 | 06 | 07 | 08 |
| Ingredients | % w/w | % w/w | % w/w | % w/w | % w/w | % w/w | % w/w | % w/w |
| Poloxamer 407 | 0.4 | 0.5 | 0.3 | 0.3 | 1 | 1 | 1 | 0.5 |
| CMCNa | | | 0.1 | 0.2 | 0.825 | 0.925 | 0.925 | 0.425 |
| Polyvinyl alcohol | 1.425 | 1.425 | 1.425 | 1.425 | | | | 1 |
| Oxovasin | 98 | 98 | 98 | 98 | 98 | 98 | 98 | 98 |
| DCAM | 0.1 | | 0.1 | | 0.1 | | | |
| Sodium carbonate | 0.075 | 0.075 | 0.075 | 0.075 | 0.075 | 0.075 | 0.075 | 0.075 |

TABLE 5
Foamable compositions (Oxo100430-01 through Oxo100430-07)

| | Formulations | | | | | | |
|---|---|---|---|---|---|---|---|
| | Oxo10430- | Oxo10430- | Oxo10430- | Oxo10430- | Oxo10430- | Oxo10430- | Oxo10430- |
| | 01 | 02 | 03 | 04 | 05 | 06 | 07 |
| Ingredients | % w/w | % w/w | % w/w | % w/w | % w/w | % w/w | % w/w |
| Poloxamer 407 | 0.5 | | | 1.25 | 0.25 | 0.5 | 0.5 |
| HPC HY117 | | 0.5 | 1 | 0.675 | 0.25 | 0.5 | 0.5 |
| Polyvinyl alcohol | 1.425 | 1.425 | | | 1.425 | 0.925 | 1.425 |
| Oxovasin | 98 | 98 | 98.925 | 98 | 98 | 98 | 97.5 |
| Sodium carbonate | 0.075 | 0.075 | 0.075 | 0.075 | 0.075 | 0.075 | 0.075 |

TABLE 6
Foamable compositions (F1 through F6)

| | Formulations | | | | | |
|---|---|---|---|---|---|---|
| Ingredients | F1 | F2 | F3 | F4 | F5 | F6 |
| Tween 20 | 0.5 | 0.5 | | | | |
| Tween 40 | | | 0.5 | 0.5 | | |
| Tween 80 | | | | | 0.5 | 0.5 |
| HPC HY117 | 0.5 | 1 | 0.5 | 1 | 0.5 | 1 |
| Na2CO3 | 0.5 | 0.5 | 0.5 | 0.5 | 0.5 | 0.5 |
| Oxovasin | 98.5 | 98 | 98.5 | 98 | 98.5 | 98 |

TABLE 7
Foamable compositions (Ox100712-01 through Ox100712-05).

| | Formulations | | | | |
|---|---|---|---|---|---|
| | Ox100712- | Ox100712- | Ox100712- | Ox100712- | Ox100712- |
| | 01 | 02 | 03 | 04 | 05 |
| Ingredients | % w/w | % w/w | % w/w | % w/w | % w/w |
| Poloxamer 407 | 0.5 | 1.25 | 0.25 | | 0.25 |
| Methocel ™ | | 0.66 | 0.25 | 0.5 | |
| Polyvinyl | 1.41 | | 1.41 | 1.41 | 1.41 |
| alcohol 99% | | | | | |
| hydrolyzed | | | | | |
| Oxovasin ™ | | 98 | | | |
| OXO-K993 | 1.96 | | 1.96 | 1.96 | 1.96 |
| Water | 96.04 | | 96.04 | 96.04 | 96.04 |
| Sodium | 0.09 | 0.09 | 0.09 | 0.09 | 0.09 |
| carbonate | | | | | |
| Natrasol | | | | | 0.25 |

TABLE 8
Foamable compositions (Ox100520-02 through Ox100520-04).
Ox100520-03 and Ox100520-04 formulations are identical to
formulations Oxo100430-05 and Oxo100430-06, respectively,
as set out in Table 5.

| | Formulations | | |
|---|---|---|---|
| | Ox100520- | Ox100520- | Ox100520- |
| | 02 | 03 | 04 |
| Ingredients | % w/w | % w/w | % w/w |
| Poloxamer 407 | 1.25 | 0.25 | 0.5 |
| HPC HY117 | 0.675 | 0.25 | 0.5 |
| Polyvinyl alcohol (80% | | 1.425 | 0.925 |
| hydrolyzed) | | | |
| Oxovasin ™ | 98 | 98 | 98 |
| Sodium carbonate | 0.075 | 0.075 | 0.075 |

TABLE 9
Foamable compositions (Ox100802-01 through Ox100802-03).
Formulation Ox100802-01 is similar to formulation
Ox100712-01 as set out in Table 7.

| | Formulations | | |
|---|---|---|---|
| | Ox100802- | Ox100802- | Ox100802- |
| | 01 | 02 | 03 |
| Ingredients | % w/w | % w/w | % w/w |
| Poloxamer 407 | 0.5 | 0.25 | |
| HPC HY 117 | | 0.25 | 0.5 |
| Polyvinyl alcohol 99% | 1.41 | | 1.41 |
| TCDO | 1.96 | 1.96 | 1.96 |
| Water | 96.04 | 96.04 | 96.04 |
| Sodium carbonate | 0.09 | 0.09 | 0.09 |

TABLE 10
pH stability of formulations containing Oxovasin ™,
hydroxypropyl cellulose, sodium carbonate (0.5%) and
either polysorbate 20 (0.5%) or polysorbate 40 (0.5%).

Weeks
Elapsed

| | | |
|---|---|---|
| | 0 | 1 |
| Replicate | pH | pH |

Oxovasin ™ with polysorbate 20 and HPY 117 0.5%

| | | |
|---|---|---|
| 1 | 11.07 | 10.97 |
| 2 | 11.12 | 11.01 |
| 3 | 11.15 | 11.03 |

Oxovasin ™ with polysorbate 20 and HPY 117 1%

| | | |
|---|---|---|
| 1 | 10.97 | 10.97 |
| 2 | 11.04 | 11.04 |
| 3 | 11.07 | 11.07 |

Oxovasin ™ with polysorbate 40 and HPY 117 0.5%

| | | |
|---|---|---|
| 1 | 11.09 | 11.00 |
| 2 | 11.13 | 11.06 |
| 3 | 11.16 | 11.10 |

Oxovasin ™ with polysorbate 40 and HPY 117 1%

| | | |
|---|---|---|
| 1 | 11.32 | 10.91 |
| 2 | 11.33 | 11.10 |
| 3 | 11.26 | 11.17 |

Oxovasin ™ with polysorbate 20 and HPY 117 0.5%

| | | |
|---|---|---|
| 1 | 11.31 | 11.19 |
| 2 | 11.28 | 11.20 |
| 3 | 11.35 | 11.20 |

Oxovasin ™ with polysorbate 20 and HPY 117 1%

| | | |
|---|---|---|
| 1 | 11.26 | 11.17 |
| 2 | 11.21 | 11.17 |
| 3 | 11.27 | 11.19 |

1.-56. (canceled)

57. A foamable composition comprising: (i) stabilized chlorite; (ii) at least one foaming agent and, optionally, at least one foam stabilizer; (iii) water; and, optionally, (iv) a buffering agent.

58. The foamable composition of claim 57, comprising at least one foam stabilizer.

59. The foamable composition of claim 57, comprising: (i) about 0.1% to about 10% (w/w) of stabilized chlorite; (ii) about 0.5% to about 5% (w/w) of at least one foaming agent; (iii) about 0.0% to about 1.0% (w/w) of a buffering agent; and (iv) water (q.s.).

60. The foamable composition of claim 59, wherein the stabilized chlorite is present in the composition in an amount of about 0.5% to about 3% (w/w); the at least one foaming agent is present in the composition in an amount of about 1% to about 3% (w/w); water is present in the composition in an amount of about 85% to about 98.5% (w/w); and the buffering agent is present in the composition in an amount of about 0.0% to about 0.2% (w/w).

61. The foamable composition of claim 57, comprising about 0.01% (w/w) to about 1.0% (w/w) of chlorite ions.

62. The foamable composition of claim 59, wherein the at least one foaming agent, includes, in addition to the foaming agent, at least one foam stabilizer.

63. The foamable composition of claim 57, wherein the stabilized chlorite is OXO-K993, or the stabilized chlorite and water combined comprise a solution of OXO-K993.

64. The foamable composition of claim 63, wherein the stabilized chlorite and water combined comprise a stabilized chlorite solution that is a 1-10%, a 10-20%, a 20-30%, a 30-50% or a 50-90% (w/v) diluted solution of OXO-K993.

65. The foamable composition of claim 64, wherein the stabilized chlorite and water combined comprise a stabilized chlorite solution that is a 2% (w/v) solution of OXO-K993.

66. The foamable composition of claim 57, wherein the foaming agent and optional foam stabilizer are selected from the group consisting of poloxamer copolymers, polyvinylalcohol (PVA), polyvinylpyrrolidone (PVP), polysorbates, cellulose ethers and coco-derived amphoteric surfactants, and combinations thereof.

67. The foamable composition of claim 66, wherein the poloxamer copolymer is selected from the group consisting of poloxamer 188, poloxamer 407, poloxamer 124, poloxamer 338, poloxamer 237, and mixtures thereof.

68. The foamable composition of claim 66, wherein, the cellulose ether is selected from the group consisting of hydroxypropycellulose (HPC), methyl cellulose, hydroxyethylcellulose (HEC), hydroxypropylmethylcellulose (HPMC), and hydroxyethylmethylcellulose (HEMC), and mixtures thereof.

69. The foamable composition of claim 57, wherein the foaming agent and optional foam stabilizer comprise, consist or consist essentially of a poloxamer, a cellulose ether and, optionally, PVA.

70. The foamable composition of claim 57, wherein the buffering agent is an inorganic or organic base or a salt of an organic base and wherein the inorganic base or salt of an organic is an alkali metal base.

71. The foamable composition of claim 70, wherein the alkali metal base is selected from the group consisting of sodium carbonate, pentasodium triphosphate, potassium pyrophosphate, sodium pyrophosphate, sodium citrate, potassium citrate, potassium carbonate, sodium bicarbonate, potassium bicarbonate, and mixtures thereof.

72. The foamable composition of claim 57, wherein the composition has a pH in the range of about 9.5 to about 12.5, about 9.5 to about 11.5, about 9.5 to about 10.5, or about 9.5 to about 10.

73. The foamable composition of claim 57, comprising:
about 97.5% to about 98.925% (w/w) of stabilized chlorite and water;
about 1% to 2.5% (w/w) of a foaming agent and, optionally, a foam stabilizer, selected from the group consisting of a poloxamer, HPC and, optionally, PVA, and mixtures thereof; and
about 0.05% to about 0.1% (w/w) of an inorganic base.

74. A foam comprising a foamable composition of claim 57.

75. A method of treating a condition for which topical or transdermal administration of stabilized chlorite is beneficial comprising administering an effective amount of the foamable composition of claim 57 to a subject in need thereof.

76. A method of treating a condition for which topical or transdermal administration of stabilized chlorite is beneficial comprising administering an effective amount of the foam of claim 74 to a subject in need thereof.
